(19) United States
(12) Patent Application Publication
(10) Pub. No.: US 2020/0315914 A1
(43) Pub. Date: Oct. 8, 2020

(54) MEDICAL VESSELS AND SYSTEMS WITH PIERCING MEMBERS AND UNLOCK MECHANISMS

(71) Applicant: Mikael NAYGAUZ, Forrest Hills, NY (US)

(72) Inventor: Mikael NAYGAUZ, Forrest Hills, NY (US)

(21) Appl. No.: 16/504,286

(22) Filed: Jul. 7, 2019

Related U.S. Application Data
(60) Provisional application No. 62/830,453, filed on Apr. 7, 2019.
(60) Provisional application No. 62/851,454, filed on May 22, 2019.
(60) Provisional application No. 62/857,260, filed on Jun. 4, 2019.
(60) Provisional application No. 62/863,210, filed on Jun. 18, 2019.

(51) Int. Cl. A61J 1/20; A61J 1/14

(52) U.S. Cl. CPC A61J 1/2006; A61J 1/1406; A61L 2202/121; A61J 1/2065; A61J 1/1481; A61J 1/1443

(57) ABSTRACT

The present invention relates, in some embodiments thereof, to devices, systems and methods for establishing a fluid passageway between vessels. In some embodiments, the invention provides a system for the engagement and establishment of fluid passageway between vessels, the system comprises a first and second vessels configured to be coupled to each other wherein at least one of the first and second vessels comprises a piercing member disposed within the vessel in a retracted position wherein the piercing member confined within/adjacent a port or aperture of the vessel, the piercing member configured to pierce through a cover/seal of the vessel at about the time of or following the engagement between the first and second vessels, thereby establishing a fluid transfer between the vessels.

### CROSS REFERENCE TO RELATED APPLICATIONS

The present application claims priority to U.S. Provisional Patent Application Ser. No. 62/830,453, entitled SYSTEM AND METHOD FOR UNLOCKING PIERCING MEMBERS OF CONTAINERS and filed on Apr. 7, 2019; 62/851,454, entitled PLUNGER AND NEEDLE LOCKING AND UNLOCKING SYSTEMS and filed on May 22, 2019; 62/857,260, entitled MEDICAL VESSELS WITH VALVES AND VALVES LOCK-UNLOCK MECHANISMS and filed on Jun. 4, 2019; and 62/863,210 entitled MEDICAL VESSELS AND SYSTEMS WITH PIERCING MEMBER AND UNLOCK MECHANISMS THEREOF and filed on Jun. 18, 2019 the entire contents of which are expressly incorporated herein by reference.

All of the following U.S. patent applications are hereby incorporated herein by reference in their entireties: Ser. Nos. 16/100,840; 16/100,860; 16/100,874, 16/100,903; 16/169,695; 16/100,964; 16/100,969; 16/100,976; 16/100,981; 16/358,434; 16/358,389; 16/423,120; 16/423,122; 16/439,851; and Ser. No. 16/254,576.

### FIELD OF THE INVENTION

The present invention relates, in some embodiments thereof, to devices, systems and methods for establishing a fluid passageway between vessels. In some embodiments, the invention provides a system for the engagement and leak-free establishment of fluid passageway(s) between vessels.

### BACKGROUND OF THE INVENTION

The medicinal practice routinely involves administration of medical substances, such as, medicaments, fluids, nutrition substances and the alike, to patients. The preparation and/or administration of such medicinal substances typically involves one or more transfers of those substances between vessels (such as, vial, syringes, infusion lines, etc.).

Additionally, beneficial substances used in medical settings such as medicaments, nutritional substances, and the alike are provided in standard commercially available amounts. This poses a disadvantage for preparing tailored and/or customized final dosage amounts for patients. The preparation of customized amounts of intracorporeally administered beneficial substances requires the manual manipulation of the beneficial substances typically by healthcare personnel. Manual manipulation involves drawing out from vials using syringes and needles of amounts of beneficial substances, measuring visually the amount of the beneficial substances that has been drawn into a syringe, and injecting the amount of the beneficial substance from a syringe into a second container, typically a bag or a bottle. The preparation of customized amount of beneficial substances typically involves using less or more than the amount provided in one commercially available container. Customized dosages particularly of chemotherapeutic agents, are typically based on a patient's weight, height, and age. Therefore, the preparation of customized final amounts of beneficial substances require the manual manipulation and measurement of beneficial substances by healthcare personnel.

Another prominent problem that exists when intermixing beneficial substances relates to the premature and/or accidental expulsion/spillage of a beneficial substance out of a container/device prior establishment of a fluid communication between containers/devices. For example, pharmacists when mixing an injectable medicament may draw out a dose of the medicament from a vial into a syringe. Pharmacists may accidentally push the plunger of the syringe before the syringe is engaged/connected to a second container such as a bag/bottle which would result in the expulsion/spillage of the medicament out of the syringe and into the environment and exposure of the pharmacist to the medicament. This is particularly dangerous if the medicament is a toxic substance such as a chemotherapeutic medicament which would result in the pharmacist being exposed to a toxic substance.

Another prominent problem that exists when intermixing beneficial substances relates to how a fluidic communication is established between containers and/or devices. For example, a pharmacist, nurse, or other healthcare provider may use a syringe having a needle to draw out a medicament from a vial. The needle is pushed through a bung (may be called a stopper), the bung may be a rubber bung (rubber stopper). The needle of the syringe is pushed through the bung to access the fluid/medicament located inside the vial. The airtight seal between the syringe and the vial is mediated via the external portion of the syringe's needle being surrounded in an airtight manner by the bung (rubber bung) of the vial. However, the airtight surrounding of the syringe's needle by the vial's rubber stopper is not guaranteed. Frequently in clinical practice the pharmacist/nurse pushes/moves the needle slightly to the left or to the right, and/or sometimes at an angle in relation to the rubber bung of the vial which may produce a small space between the needle and the vial's bung through which small quantities of the beneficial substance/medicament may escape the vial (between the space between the syringe's needle and the vial's rubber bung). Optionally, any vapors contained within the vial may escape between the space between the needle and the vial bung. This occurs frequently and illustrates that the fluidic communication between containers and/or between a container (vial) and a medical device (syringe with needle) is prone to leakage. Such leakage of a beneficial substance and/or vapors from within a container (such as a vial) into the environment exposes a user (pharmacist, nurse, etc.) to the contents contained within the vial.

Yet another problem is associated with contamination of medical substances when intermixing beneficial substances. Typically, the action of fluid transfer exposes connection interfaces/surfaces of medical vessels to the environment and accordingly the medical substances themselves to contaminants present in ambient air particles (e.g., bacteria, viruses, funguses, spores, pyrogens). In addition, connection interfaces/surfaces of medical substances are further prone to contaminations due to physical contact of the interfaces/surfaces, for example, with nonsterile gloves, or devices. Such contaminations are a major problem in the healthcare setting since those, once invading within medicinal substances, may pose substantial danger to patients.

Taken together, the above described systems and methods are either cumbersome and expensive or inefficient in addressing the problem of intermixing customized amounts of beneficial substances while preventing the premature/accidental expulsion/spillage of the beneficial substances prior to establishment of a fluidic communication between containers/devices. Thus, there is a long felt and unmet need in systems, devices and/or methods that afford the safe transfer of medical substances while preventing the spillage of medical substances and exposure of healthcare personnel to toxic substances.

### SUMMARY OF THE INVENTION

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

The present invention pertains to devices, systems and methods for establishing a fluid communication between vessels in a secure manner and while allowing a fluidic transfer when vessels attach to each other. It is an object of the present invention, in some embodiments, to provide systems, devices, containers, and methods of preventing spillage of beneficial substances/medicaments prior to engagement of vessels.

Thus, the present invention provides a system for the engagement and establishment of fluid passageway between vessels, the system comprises:

a first vessel; and

a second vessel configured to be coupled to the first vessel;

wherein at least one of the first and second vessels comprises a piercing member, the piercing member disposed within the vessel in a retracted position and configured to pierce through a cover/seal of the vessel at about the time of or following the engagement between the first and second vessels, thereby establishing a fluid transfer between the vessels.

In another aspect, the present invention provides a system for the engagement and establishment of fluid passageway between vessels, the system comprises:

a first vessel;

a second vessel; and

a decontamination device configured to be coupled to the first vessel and second vessel and allow a fluid transfer therebetween, the decontamination device further configured to decontaminate a surface of the first and/or second vessels;

wherein at least one of the first vessel, the second vessel and the decontamination device comprises a piercing member, the piercing member disposed within the vessel and/or decontamination device in a retracted position and configured to pierce through a cover/seal of the vessel and/or decontamination device at about the time of or following the engagement between the first and/or second vessels and the decontamination device, thereby establishing a fluid transfer between the vessels.

In one or more embodiments, the piercing member is configured to pierce through a cover/seal of both the first and second vessels at about the time of or following the engagement between the first and second vessels.

In one or more embodiments, the piercing member is configured to pierce through a cover/seal of the decontamination device at about the time of or following the engagement between the first and second vessels.

In one or more embodiments, the system further comprising a piercing member lock mechanism connected to the piercing member and configured to maintain the piercing member in the retracted position prior to an engagement between the vessels, thereby preventing accidental fluid spillage from the vessel.

In one or more embodiments, the piercing member lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the piercing member lock mechanism connected to the piercing member. In one or more embodiments, the piercing member lock mechanism disposed in the first vessel, the second vessel and/or the decontamination device.

In one or more embodiments, the piercing member lock mechanism extends through a wall of the first vessel, the second vessel and/or the decontamination device. In one or more embodiments, the piercing member lock mechanism includes an inner portion connected to the piercing member and an outer portion protruding from a surface of the first vessel, the second vessel and/or the decontamination device. In one or more embodiments, the piercing member lock mechanism includes an inner portion connected to the piercing member and an opposing portion slotted within a wall of the first vessel, the second vessel and/or the decontamination device.

In one or more embodiments, the system further comprising a piercing member unlock mechanism configured to unlock/allow movement of the piercing member, and wherein the unlock mechanism unlocks the piercing member at about the time of or following the engagement between the first and second vessels.

In one or more embodiments, the system further comprising a piercing member unlock mechanism configured to engage/connect the lock mechanism and unlock thereof, thereby allow the movement of the piercing member, and wherein the unlock mechanism unlocks the piercing member lock mechanism at about the time of or following the engagement between the first and second vessels.

In one or more embodiments, the piercing member unlock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

In one or more embodiments, the piercing member pierces through a seal/cover of a vessel via a push motion, a twist motion, a turn motion or a combination thereof between the first and second vessels.

In one or more embodiments, the system further comprising an engagement mechanism to connect the first vessel to the second vessel.

In one or more embodiments, the engagement mechanism is selected from a thread, a luer, a ratchet teeth mechanism, a retention member, an adhesive mechanism, a clamping member and combinations thereof.

In one or more embodiments, the system further comprising a valve within one or both vessels to monitor the fluid transfer between the first and/or second vessel(s).

In one or more embodiments, the system further comprising a valve lock mechanism and optionally a valve unlock mechanism to prevent accidental fluid spillage from a vessel.

In one or more embodiments, the system further comprising a plunger disposed on the first and/or second vessel(s), the plunger configured to allow for the fluid transfer between the first vessel and the second vessel.

In one or more embodiments, the system further comprising a plunger lock mechanism configured to lock a plunger and prevent fluid transfer between the first vessel and second vessels prior to an engagement between the vessels.

In one or more embodiments, the vessel is selected from the group consisting of a bottle, a syringe, an infusion line, a connector, a filter, a manifold, a bag port, a bottle port, a vial port, a cartridge and a combination thereof.

In one or more embodiments, the system further comprising a decontamination device configured to be coupled to the first vessel and second vessel and allow the fluid passageway therebetween, the decontamination device configured to decontaminate a surface of at least one of the first and second vessels.

In one or more embodiments, the system further comprising an engagement mechanism to connect the decontamination device to the first and/or second vessel.

In one or more embodiments, the vessel and/or decontamination device includes a port having peripheral vertical walls enclosing a lumen, the lumen configured to receive or engage with a vessel and/or decontamination device, or a portion thereof.

In one or more embodiments, the decontamination device is pre-attached to the first or second vessels.

In one or more embodiments, the decontamination device is integrally attached to the first or second vessel.

In one or more embodiments, the decontamination device includes a wiping member configured to wipe off a surface of a vessel.

In one or more embodiments, the first and/or second vessel is configured to move within the device and wherein the wiping member configured to remove contaminants from a surface of said first and/or second vessels at about the time of said vessel's movement.

In one or more embodiments, the wiping member is configured to move within the device such to allow decontamination of a surface of at least one of said first and second vessels at about the time of said wiping member's movement.

In one or more embodiments, the decontamination device includes a sliding mechanism located on and/or attached to said wiping member and/or said vessel and configured to allow for the movement of said wiping member and/or said vessel within said device.

In one or more embodiments, the system further comprising a piercing member disposed within the decontamination device and configured to pierce through a seal or cover of a vessel and/or port of the decontamination device and prevent the premature fluid transfer between the first vessel and the second vessel and/or fluid spillage, prior to an engagement between the first and/or second vessels and the decontamination device.

In one or more embodiments, the second vessel includes a port having peripheral walls enclosing a cavity, the cavity configured to receive or engage with the first vessel, or a portion thereof.

In one or more embodiments, the first vessel is a cartridge. In one or more embodiments, the second vessel includes a port having peripheral walls enclosing a cavity, the cavity is sized and shaped to accommodate the cartridge.

In yet another aspect the invention provides a method for establishing a fluidic communication between vessels, the method comprising:

providing a first vessel and a second vessel, wherein the first and/or second vessel includes a piercing member, the piercing member disposed within the vessel when in a retracted position;

engaging/coupling the first and second vessels, wherein at about the time of or following the engagement between the vessels the piercing member is unlocked, allowing movement of the piercing member and piercing through a seal/cover of the first and/or second vessel, thereby establishing a fluidic communication between the first and second vessels.

In one or more embodiments, an airtight seal is formed between said vessels following the step of connecting/engaging the vessels, allowing a hermetically sealed connection between said first vessel and said second vessel.

In one or more embodiments, in the retracted position the piercing member is locked by a piercing member lock mechanism and wherein the step of connecting/engaging the vessels includes unlocking the lock mechanism.

In one or more embodiments, unlocking the lock mechanism is conducted via a piercing member unlock mechanism.

In one or more embodiments, the step of connecting/engaging the vessels includes unlocking the lock mechanism via a piercing member unlock mechanism.

In one or more embodiments, the step of unlocking the piercing member occurs via a push motion, a twist motion, a turn motion or a combination between the first and second vessels. In one or more embodiments, the connection/engagement of the vessels occurs via a push motion, a twist motion, a turn motion or a combination thereof between the first and vessels.

In one or more embodiments, the connection/engagement of said vessels is selected from a twisting of a thread onto a complementary thread, attaching ratchet teeth to a complementary retention member, and adhering a surface of a first vessel to the surface of said second vessel via an adhesive, and combinations thereof.

In one or more embodiments, the method further comprising pushing a plunger of the first or second vessel towards the respective vessel.

In one or more embodiments, the method further comprising providing a decontamination device having a wiping member, the wiping member configured to decontaminate a surface of the first and/or second vessel(s).

In one or more embodiments, the method further comprising actuating the wiping member to move within the device, thereby decontaminating the surface of the first and/or second vessel.

In one or more embodiments, the method further comprising moving the first vessel and/or second vessel within the device and via the wiping member, thereby decontaminating the surface of the first and/or second vessel.

In one or more embodiments, the method further comprising transferring a fluid substance between the vessels after engaging/connecting the vessels.

In one or more embodiments, the method further comprising transferring a fluid substance within the at least one vessel through the device after the surface of the at least one vessel has been decontaminated.

In yet another aspect the present invention provides a container having a piercing member and a piercing member lock mechanism.

In one or more embodiments, the piercing member further comprising a dent configured to allow entry of a portion of the lock mechanism.

In one or more embodiments, the lock mechanism is characterized in that it is at least partially disposed inside the container and at least partially exposed exterior to the container.

In one or more embodiments, the lock mechanism is characterized in that it is entirely within the contained.

In one or more embodiments, the lock mechanism is at least partially disposed inside a wall of the container and at least partially disposed inside the container.

In one or more embodiments, the lock mechanism is attached to the container via a hinge mechanism.

In one or more embodiments, the lock mechanism is attached to the container via a hinge.

In one or more embodiments, the container is a syringe.

In one or more embodiments, the container is a cartridge.

In one or more embodiments, the container is a bottle.

In one or more embodiments, the container is a bag.

In one or more embodiments, the lock mechanism is configured to actuate the movement of the piercing member through a wall of the container.

In one or more embodiments, the lock mechanism is configured to actuate the movement of the piercing member through a seal of the container.

In one or more embodiments, the locking mechanism is a pin mechanism.

In yet a further aspect, the present invention provides a decontamination device having a piercing member unlocking mechanism.

In one or more embodiments, the decontamination device comprising a housing having a wiper disposed within the housing.

In one or more embodiments, the wiper is configured to move within the housing.

In one or more embodiments, the decontamination device further comprising an actuator disposed through a wall of the housing, the actuator configured to mediate the movement of the wiper within the housing.

In one or more embodiments, the decontamination device having a wiper disposed within the housing, the device further comprising a rail mechanism configured to allow movement of a container/device along the housing.

In yet a further aspect, the present invention provides a decontamination device having a piercing member and a piercing member locking mechanism.

In one or more embodiments, the piercing member locking mechanism configured to be unlocked by a piercing member unlocking mechanism of another container.

In one or more embodiments, the piercing member locking mechanism configured to be unlocked by a piercing member unlocking mechanism of another device.

In one or more embodiments, the piercing member locking mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the piercing member unlocking mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In yet a further aspect, the present invention provides a system for the establishment of a fluidic communication between containers, comprising:

a first container having a piercing member and a piercing member lock mechanism; and

a second container,

wherein when the first container couples with the second container, the second container unlocks the piercing member lock mechanism of the first container thus allowing the piercing member of the first container to pierce through a surface of at least one of the first and second containers.

In one or more embodiments, the first container is a bag.

In one or more embodiments, the second container is a bag.

In one or more embodiments, at least one of the first and second containers is a bottle.

In one or more embodiments, the first container is a bag and the second container is a bottle.

In one or more embodiments, the first container is a bag and the second container is a syringe.

In one or more embodiments, the first container is a bag and the second container is a cartridge.

In one or more embodiments, the piercing member lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the piercing member lock mechanism is disposed inside a wall of the first container.

In yet a further aspect, the present invention provides a system for the establishment of a fluidic communication between containers, comprising:

a first container having a piercing member and a piercing member lock mechanism; and

a second container having a piercing member unlock mechanism,

wherein when the first container couples with the second container, the second container's piercing member unlock mechanism unlocks the piercing member lock mechanism of the first container thus allowing the piercing member of the first container to pierce through a surface of at least one of the first and second containers.

In one or more embodiments, the first container is a bag.

In one or more embodiments, the second container is a bag.

In one or more embodiments, at least one of the first and second containers is a bottle.

In one or more embodiments, the first container is a bag and the second container is a bottle.

In one or more embodiments, the first container is a bag and the second container is a syringe.

In one or more embodiments, the first container is a bag and the second container is a cartridge.

In one or more embodiments, the piercing member lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the piercing member lock mechanism is disposed inside a wall of the first container.

In yet a further aspect, the present invention provides a container having a piercing member disposed inside the container.

In one or more embodiments, the piercing member is entirely disposed inside the container.

In one or more embodiments, the container is a bag.

In one or more embodiments, the container is a bottle.

In one or more embodiments, the container is a syringe.

In one or more embodiments, the container is a cartridge.

In one or more embodiments, the container further comprises a piercing member locking mechanism.

In one or more embodiments, the piercing member locking mechanism is selected from a latch, a pin, a dent, a ball, a retractable ball, and combinations thereof.

One or more embodiments, the piercing member is disposed inside a wall of the container.

In yet a further aspect, a method is provided for the establishment of a leak-free fluidic communication between containers, the method comprising:
providing a first container having a piercing member disposed inside the container;
providing a second container;
engaging the first container to the second container; and
piercing a surface of the first and second containers by the piercing member.

In one or more embodiments, the piercing of the surface of the first and second containers occurs after the engagement of the first and second containers.

In one or more embodiments, the first and second containers are engaged prior to piercing of the surface of the first and second containers by the piercing member.

In one or more embodiments, the piercing member is a needle.

In one or more embodiments, the needle is a hollowed needle.

In one or more embodiments, the first container is a bag and the second container is bottle.

In one or more embodiments, the first container is a bag and the second container is a syringe or a vial.

In one or more embodiments, the first container is a bag and the second container is a cartridge.

In one or more embodiments, the first container is a bottle and the second container is a bottle or a syringe.

In one or more embodiments, the first container is a bottle and the second container is a vial or a cartridge.

In one or more embodiments the first container further comprises a piercing member lock mechanism.

In one or more embodiments, the second container further comprises a piercing member unlock mechanism configured to unlock the piercing member lock mechanism of the first container.

In one or more embodiments, the piercing member lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the piercing member unlock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In yet a further aspect a method is provided for the establishment of a fluidic communication between vessels, the method comprising:
providing a first vessel having a piercing member and a piercing member lock mechanism;
providing a decontamination device;
providing a second vessel;
connecting the first vessel to the decontamination device; and
connecting the second vessel to the decontamination device.

In one or more embodiments, the method further comprises decontaminating a surface of at least one of the first and second vessels.

In one or more embodiments, the decontamination device has a wiping member.

In one or more embodiments, the decontamination device has a wiping member disposed inside the decontamination device.

In one or more embodiments, the decontamination device has a wiping member at least partially disposed inside the decontamination device.

In one or more embodiments, the first and the second vessels are containers.

In yet a further aspect a method is provided for the establishment of a fluidic communication between vessels, the method comprising:

providing a first vessel having a decontamination device, a piercing member, and a piercing member lock mechanism;

providing a second vessel;

engaging the second vessel to the decontamination device of the first vessel; and

decontaminating a surface of at least one of the first and second vessels.

In one or more embodiments, the method further comprising unlocking of the piercing member lock mechanism of the first vessel.

In one or more embodiments, the method further comprising unlocking of the piercing member lock mechanism of the first vessel by the second vessel.

In one or more embodiments, the second vessel further comprising a piercing member unlock mechanism.

In one or more embodiments, the method further comprising unlocking of the first vessel's piercing member lock mechanism by the piercing member unlock mechanism of the second vessel.

In one or more embodiments, the first and second vessels are containers.

In one or more embodiments, the first vessel is a container and the second vessel is a syringe.

In one or more embodiments, the first vessel is a bag and the second vessel is a cartridge.

In one or more embodiments, the first vessel is a bottle and the second vessel is a cartridge.

In one or more embodiments, the first vessel is a bag and the second vessel is a syringe.

In one or more embodiments the first vessel is a container and the second vessel is a bottle.

In one or more embodiments, the first and second containers are bottles.

In one or more embodiments, the first vessel is a bag and the second vessel is a vial.

In one or more embodiments, the first vessel is a bottle and the second vessel is a vial.

In one or more embodiments, the first vessel is a vial and the second vessel is a container.

In one or more embodiments, the first vessel is a cartridge and the second vessel is a container.

In one or more embodiments, the first vessel is a syringe and the second vessel is a container.

In one or more embodiments, the decontamination device having a wiping member configured to wipe off a surface of at least one of the first and second vessels.

In one or more embodiments, the method comprising wiping off a surface of at least one of the first and second vessels prior to piercing of a surface of at least one of the first and second vessels.

In one or more embodiments, the method further comprising piercing of a surface of at least one of the first and second vessels after the decontamination of at least one of the first and second vessel surfaces.

In yet a further aspect a system is provided for the establishment of a contaminant-free fluidic communication between vessels, the system comprising:

providing a first vessel having a piercing member and a piercing member lock mechanism;

providing a second vessel; and

providing a decontamination device.

In one or more embodiments, the second vessel further comprising a piercing member unlocking mechanism configured to unlock the piercing member lock mechanism of the first vessel.

In one or more embodiments, the decontamination device is integrally attached to the first or the second vessels.

In one or more embodiments, the first and second vessels are containers.

In one or more embodiments, at least one of the first and second vessels is a container and at least one of the first and second vessels is a syringe.

In one or more embodiments, at least one of the first and second vessels is a container and at least one of the first and second vessels is a cartridge.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B are side cut view illustrations demonstrating a syringe with a piercing member disposed within a tip of the syringe and a piercing member lock mechanism configured prevent premature puncture of the syringe's seal, according to some embodiments of the invention.

FIGS. 2A-2B are side cut view illustrations demonstrating a syringe with a piercing member and a lock mechanism thereof, the lock mechanism having a latch-like form, according to some embodiments of the invention.

FIGS. 3A-3B are side cut view illustrations demonstrating a syringe with a piercing member and a lock mechanism thereof, the piercing member mounted to the syringe via a supporting base, according to some embodiments of the invention.

FIGS. 4A-4B are side cut view illustrations demonstrating a syringe with a piercing member and a lock mechanism thereof, the lock mechanism connected to the piercing member via a slot within the piercing member, according to some embodiments of the invention.

FIGS. 5A-5B are side cut view illustrations demonstrating a syringe with a piercing member and a lock mechanism thereof, the lock mechanism connected to the piercing member via a dent within the piercing member, according to some embodiments of the invention.

FIGS. 6A-6B are side cut view illustrations demonstrating a syringe with a piercing member and a lock mechanism thereof, the lock mechanism having a latch-like end which grasps a protrusion within/on the piercing member, according to some embodiments of the invention.

FIG. 7 is a side cut view illustration demonstrating a syringe with a piercing member and a lock mechanism thereof, the lock mechanism connected to the piercing member via a slot within the piercing member, according to some embodiments of the invention.

FIG. 8 is a side cut view illustration demonstrating a syringe with a piercing member and a lock mechanism thereof, the lock mechanism connected to the piercing member via a slot within the piercing member, according to some embodiments of the invention.

FIGS. 9A-9B are side cut view illustrations demonstrating a cartridge with a piercing member disposed within the cartridge and a piercing member lock mechanism configured prevent premature puncture of the cartridge's seal, according to some embodiments of the invention.

FIGS. 10A-11B are side cut view illustrations demonstrating a cartridge with a piercing member and a lock mechanism thereof, the lock mechanism having a latch-like form, according to some embodiments of the invention.

FIGS. 11A-11B are side cut view illustrations demonstrating a cartridge with a piercing member and a lock mechanism thereof, the piercing member mounted to the cartridge via a supporting base, according to some embodiments of the invention.

FIGS. 12A-12B are side cut view illustrations demonstrating a cartridge with a piercing member and a lock mechanism thereof, the lock mechanism connected to the piercing member via a slot within the piercing member, according to some embodiments of the invention.

FIGS. 13A-13B are side cut view illustrations demonstrating a cartridge with a piercing member and a lock mechanism thereof, the lock mechanism connected to the piercing member via a dent within the piercing member, according to some embodiments of the invention.

FIGS. 14A-14B are side cut view illustrations demonstrating a cartridge with a piercing member and a lock mechanism thereof, the lock mechanism having a latch-like end which grasps a protrusion within the piercing member, according to some embodiments of the invention.

FIGS. 15A-15B are side cut view illustrations demonstrating a system having a first vessel, and a second vessel, the first vessel in the form of a syringe having a piercing member and a lock mechanism thereof, according to some embodiments of the invention.

FIGS. 16A-16B are side cut view illustrations demonstrating a system having a first vessel, and a second vessel, the first vessel in the form of a cartridge and the second vessel includes a piercing member and a lock mechanism thereof, according to some embodiments of the invention.

FIG. 17 is a side cut view illustration demonstrating a system with a plurality of first vessels that can be engaged with a second vessel, the second vessel includes cavities for accommodating the first vessels and piercing members with lock mechanisms, according to some embodiments of the invention.

FIG. 18 is a side cut view illustration demonstrating a system with a plurality of first vessels that can be engaged with a second vessel, the first vessels include piercing members and lock mechanisms thereof and the second vessel includes cavities for accommodating the first vessels, the second vessel further includes piercing member unlock mechanisms within the cavities for allowing a fluid communication between the vessels, according to some embodiments of the invention.

FIGS. 19A-19B are side cut view illustrations demonstrating a system with a first vessel in the form of a syringe, a second vessel in the form of an infusion bag, and a decontamination device, the first vessel having a piercing member and the decontamination device has a piercing member unlock mechanism, according to some embodiments of the invention.

FIG. 20 is a side cut view illustration demonstrating a system with optional first vessels, optional second vessels and a decontamination device in the form of a connector, the vessels include a piercing member and a piercing member lock mechanism and the decontamination device includes a piercing member unlock mechanism, according to some embodiments of the invention.

FIG. 21 is a side cut view illustration demonstrating a system with optional first vessels, optional second vessels and a decontamination device in the form of a connector, the decontamination device includes a piercing member and a piercing member lock mechanism and the vessels include a piercing member unlock mechanism, according to some embodiments of the invention.

FIG. 22 is a side cut view illustration demonstrating a system with a decontamination device connected at a first port thereof to a first vessel and at a second port thereof to a second vessel, wherein each of the vessels includes a piercing member and a piercing member lock mechanism and the decontamination device includes a piercing member unlock mechanism, according to some embodiments of the invention.

FIGS. 23A-23B are side cut view illustrations demonstrating a system with a first vessel in the form of a bottle, a second vessel in the form of an infusion bag, and a decontamination device with a moveable wiping member, the first vessel includes a piercing member and a piercing member lock mechanism a, according to some embodiments of the invention.

FIGS. 24A-24B are side cut view illustrations demonstrating a system with a first vessel in the form of a bottle, a second vessel in the form of an infusion bag, and a decontamination device with a moveable wiping member, the decontamination device includes a piercing member and a piercing member lock mechanism, according to some embodiments of the invention.

FIGS. 25A-25B are side cut view illustrations demonstrating a system with a first vessel in the form of a bottle, a second vessel in the form of an infusion bag, and a decontamination device with a moveable wiping member, the decontamination device includes a piercing member and a piercing member lock mechanism, according to some embodiments of the invention.

FIGS. 26A-26D are side cut view illustrations demonstrating a system with a first vessel in the form of a bottle, a second vessel in the form of an infusion bag, and a decontamination device with a moveable wiping member, the decontamination device includes side walls enclosing a cavity for accommodating therein the first vessel, according to some embodiments of the invention.

FIGS. 27A-27D are side cut view illustrations demonstrating a system with a first vessel in the form of a bottle, a second vessel in the form of an infusion bag, and a decontamination device with a static wiping member, the decontamination device includes downward extending walls enclosing a lumen for accommodating therein the first vessel, according to some embodiments of the invention.

FIGS. 28A-28D are side cut view illustrations demonstrating a system with a first vessel in the form of a syringe, a second vessel in the form of an infusion bag, and a decontamination device with a static wiping member, the decontamination device includes downward extending walls enclosing a lumen for accommodating therein the first vessel, according to some embodiments of the invention.

FIGS. 29A-29D are side cut view illustrations demonstrating a system with a first vessel in the form of a bottle, a second vessel in the form of an infusion bag, and a decontamination device with a static wiping member and one or more moveable discs, the decontamination device further includes downward extending walls enclosing a lumen for accommodating the first vessel, according to some embodiments of the invention.

FIG. 30 is a side cut view illustration demonstrating a system with optional first vessels, optional second vessels and a decontamination device in the form of a connector, the vessels include a piercing member and a piercing member lock mechanism and the decontamination device includes a piercing member unlock mechanism and a static wiping member, according to some embodiments of the invention.

FIG. 31 is a side cut view illustration demonstrating a system with optional first vessels, optional second vessels and a decontamination device in the form of a connector having downward extending walls, the vessels include a piercing member and a piercing member lock mechanism and the decontamination device includes a piercing member unlock mechanism and a static wiping member, according to some embodiments of the invention.

FIG. 32 is a side cut view illustration demonstrating a system with optional first vessels, optional second vessels and a decontamination device in the form of a connector having extending walls, the decontamination device includes a piercing member, a piercing member lock mechanism and a static wiping member and the vessels include a piercing member unlock mechanism, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary piercing member lock and unlock mechanisms for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention pertains to devices, methods and systems for fluid transfer between one or more vessels in a secure manner. The present invention pertains to systems, devices, and methods for preventing the premature spillage of beneficial substances/medicaments from containers intended for use in a modular assembly format. The invention provides a solution to an unmet and long felt need in the medical settings and provides vessels with a piercing member disposed within a port or tip of the vessel and configured to move forward and pierce a seal of the vessel at about the time of or following an engagement with a second vessel. The piercing member is therefore moveable between a retracted position wherein the piercing member is concealed within the port or tip of a vessel and an extended position wherein the piercing member moved forward within the tip or port of the vessel and capable of piercing a seal of the vessel. the herein disclosed systems optionally further include a piercing member lock mechanism configured to maintain the piercing member in the locked position/retracted position, preventing premature spillage of medical substances. The herein disclosed systems optionally include a piercing member unlock mechanism configured to unlock the lock mechanism and allowing movement of the piercing member in the vessel. In one or more embodiments, the herein disclosed devices and systems pertain to piercing members, piercing members lock mechanisms and piercing member unlock mechanisms for securing and monitoring a fluid transfer between vessels.

Thus, it is an object of the present invention, in some embodiments, to provide systems, devices, and methods for establishing fluidic communications between containers. It is an object of the present invention, in some embodiments, to provide vessels having one or more piercing members for fluid transfer monitoring. It is an object of the present invention, in some embodiments, to provide vessels having a piercing member locking mechanism. It is an object of the present invention, in some embodiments, to provide vessels having a piercing member unlocking mechanism. It is an object of the present invention, in some embodiments, to provide systems, devices, and methods of preventing spillage of beneficial substances/medicaments prior to the engagement of at least two vessels.

The present invention pertains to a system with at least two containers/vessels for establishing a fluidic communication therebetween at about the time, at the time, and/or following an engagement/coupling of the at least two containers/vessels. In some embodiments, the invention provides systems having a first and a second vessel, wherein the first container/vessel is having a piercing member configured to open a seal or cover of the first vessel and optionally also of the second vessel, wherein the seal's/cover's opening is induced by the engagement of the vessels, and wherein upon puncturing of the seal/cover a fluidic communication can be established between the containers/vessels. In one or more embodiments, the second vessel further includes a piercing member configured to open a seal or cover of the second vessel and optionally also of the first vessel, wherein the seal's/cover's opening is induced by the engagement of the vessels, and wherein upon puncturing of the seal/cover a fluidic communication can be established between the containers/vessels.

In one or more embodiments, engaging/coupling the vessels mediates the unlocking/movement of the piercing member, allowing for a fluidic communication to between the vessels. Advantageously, the invention provides systems capable of preventing premature leakage of contents of the containers into the environment. Hence, the system prevents exposure of healthcare personnel to optionally toxic substance, providing safe means of establishing fluidic communication between at least two containers.

The piercing member is moveable between two positions, a first retracted position in which the piercing member disposed within a tip/port/body of the vessel and a second advanced position in which the piercing member moves forward towards the seal/cover and capable of puncturing the seal/cover of the first and optionally also of the second vessel.

Thus, in one or more embodiments, the invention provides a system for establishing fluidic communication between vessels, the system comprising:

a first vessel having a piercing member; and

a second vessel;

wherein the piercing member is maintained locked within the first vessel prior to an engagement between the vessels and wherein upon the engagement between the first and second vessels, the piercing member is unlocked and capable of piercing through a seal or cover of the first vessel and optionally of the second vessel, providing a fluid communication between the vessels.

In one or more embodiments, the invention provides a system for establishing fluidic communication between vessels, the system comprising:

a first vessel having a piercing member and a piercing member lock mechanism; and

a second vessel;

wherein the piercing member configured to pierce through a seal/cover of the first vessel and optionally of the second vessel and wherein the piercing member lock mechanism configured to maintain the piercing member locked prior to an engagement with a second vessel, wherein upon the engagement between the first and second vessels, the piercing member locking mechanism is unlocked allowing movement of the piercing member and thereby piercing the seal/cover of the first vessel and optionally of the second, establishing a fluid communication between the vessels.

In one or more embodiments, the invention provides a system for establishing fluidic communication between vessels, the system comprising:

a first vessel having a piercing member and a piercing member lock mechanism; and

a second vessel;

wherein the first and/or second vessel includes a piercing member unlock mechanism, wherein upon the engagement between the first and second vessels, the piercing member unlock mechanism unlocks the piercing member locking mechanism, allowing movement of the piercing member and a fluid communication between the vessels.

In one or more embodiments, the invention provides a system for establishing fluidic communication between vessels, the system comprising:

a first vessel having a piercing member;

a second vessel; and

a decontamination device configured to be coupled to the first and second vessels, allow a fluid communication between the vessels and decontaminate a surface of the first and/or second vessels;

wherein upon the engagement between the first vessel and/or second vessel with the decontamination device, the piercing member is unlocked allowing the piercing member to pierce thorough the seal/cover of the first and optionally the second vessel, thereby providing a fluid communication between the vessels.

In one or more embodiments, the invention provides a system for establishing fluidic communication between vessels, the system comprising:

a first vessel having a piercing member and a piercing member lock mechanism;

a second vessel; and

a decontamination device configured to be coupled to the first and second vessels, allow a fluid communication between the vessels and decontaminate a surface of the first and/or second vessels;

wherein upon the engagement between the first vessel and the decontamination device, the piercing member locking mechanism is unlocked allowing the piercing member to pierce thorough the seal/cover of the first and optionally the second vessel, thereby providing a fluid communication between the vessels.

In one or more embodiments, the invention provides a system for establishing fluidic communication between vessels, the system comprising:

a first vessel having a piercing member and a piercing member lock mechanism;

a second vessel; and

a decontamination device configured to be coupled to the first and second vessels, allow a fluid communication between the vessels and decontaminate a surface of the first and/or second vessels;

wherein the first vessel and/or decontamination device include(s) a piercing member unlock mechanism, wherein upon the engagement between the first vessel and the decontamination device, the piercing member unlock mechanism unlocks the piercing member locking mechanism, allowing the piercing member to pierce thorough the seal/cover of the first and optionally the second vessel, thereby providing a fluid communication between the vessels.

In one or more embodiments, the second vessel further comprises a piercing member. In one or more embodiments, the piercing member of the second vessel is connected/engaged/coupled to a piercing member locking mechanism. In one or more embodiments, the second vessel includes a piercing member and a piercing member lock mechanism, and the first vessel includes a piercing member unlocking mechanism configured to unlock the piercing member locking mechanism of the second vessel.

The piercing member may optionally be a needle, for example, a hollowed needle, but other sharp elements are contemplated.

Various forms of the piercing member locking mechanism are contemplated and applicable, including without limitation a hook, pin, retractable pin, locking key, latch, knob, lock, dent, hole, channel, bangle, slot, or a combination thereof.

Various forms of the piercing member unlocking mechanism are contemplated and applicable, including without limitation a hook, pin, retractable pin, locking key, latch, knob, lock, dent, hole, channel, bangle, slot.

Various positions of the piercing member and/or piercing member lock mechanism and/or piercing member unlock mechanism are contemplated, such as within the vessel, within a tip or port of the vessel, on a surface of an aperture/tip/port of the vessel, on internal surfaces of side walls of the vessel. Within a cavity of the vessel, extending through a wall of the vessel, etc.

In one or more embodiments, the piercing member locking mechanism engages/couples with the piercing member unlocking mechanism at about the time of engagement/coupling of the first vessel and the second vessel. In one or more embodiments, the piercing member locking mechanism engages/couples with the piercing member unlocking mechanism at the time of engagement/coupling of the first vessel and the second vessel. In one or more embodiments, the piercing member locking mechanism engages/couples with the piercing member unlocking mechanism following the time of engagement/coupling of the first vessel and the second vessel.

In one or more embodiments, the piercing member locking mechanism engages/couples with the piercing member unlocking mechanism at about the time of engagement/coupling of the first vessel and/or the second vessel with the decontamination device. In one or more embodiments, the piercing member locking mechanism engages/couples with the piercing member unlocking mechanism at the time of engagement/coupling of the first vessel and/or the second vessel with the decontamination device. In one or more embodiments, the piercing member locking mechanism engages/couples with the piercing member unlocking mechanism following the time of engagement/coupling of the first vessel and/or the second vessel with the decontamination device.

Various motions can be applicable for unlocking the piercing member. In one or more embodiments, the unlocking of the piercing member and/or piercing member locking mechanism is mediated via a pushing motion of the second vessel towards the first vessel. In one or more embodiments, the unlocking of the piercing member and/or piercing member locking mechanism is mediated via a pushing motion of the first vessel towards the second vessel. In one or more embodiments, the unlocking of the piercing member and/or piercing member locking mechanism is mediated via a pushing motion of the first and second vessels towards each other. In one or more embodiments, the unlocking of the piercing member and/or piercing member locking mechanism is mediated via a pushing motion of the first and/or second vessel towards the decontamination device. In one or more embodiments, the unlocking of the piercing member and/or piercing member locking mechanism is mediated via a pushing motion of the decontamination device towards the first and/or second vessel. In one or more embodiments, the unlocking of the piercing member and/or piercing member locking mechanism is mediated via a pushing motion of the decontamination device and first and/or second vessel towards each other.

In one or more embodiments, the unlocking of the piercing member and/or piercing member locking mechanism is mediated via a twisting motion of the first vessel in relation to the second vessel. In one or more embodiments, the unlocking of the piercing member and/or piercing member locking mechanism is mediated via a twisting motion of the second vessel in relation to the first vessel. In one or more embodiments, the unlocking of the piercing member and/or piercing member locking mechanism is mediated via a twisting motion of the first and second vessels in relation to each other. In one or more embodiments, the unlocking of the piercing member and/or piercing member locking mechanism is mediated via a twisting motion of the first and/or second vessel towards the decontamination device. In one or more embodiments, the unlocking of the piercing member and/or piercing member locking mechanism is mediated via a twisting motion of the decontamination device towards the first and/or second vessel. In one or more embodiments, the unlocking of the piercing member and/or piercing member locking mechanism is mediated via a twisting motion of the decontamination device and first and/or second vessel towards each other.

In one or more embodiments, the unlocking of the piercing member and/or piercing member locking mechanism is mediated via a turning motion of the first vessel in relation to the second vessel. In one or more embodiments, the unlocking of the piercing member and/or piercing member locking mechanism is mediated via a turning motion of the second vessel in relation to the first vessel. In one or more embodiments, the unlocking of the piercing member and/or piercing member locking mechanism is mediated via a turning motion of the first and second vessels in relation to each other. In one or more embodiments, the unlocking of the piercing member and/or piercing member locking mechanism is mediated via a turning motion of the first and/or second vessel towards the decontamination device. In one or more embodiments, the unlocking of the piercing member and/or piercing member locking mechanism is mediated via a turning motion of the decontamination device towards the first and/or second vessel. In one or more embodiments, the unlocking of the piercing member and/or piercing member locking mechanism is mediated via a turning motion of the decontamination device and first and/or second vessel towards each other.

In one or more embodiments, the unlocking of the piercing member and/or piercing member locking mechanism is mediated via a push and twist motion of the second vessel in relation to the first vessel. In one or more embodiments, the unlocking of the piercing member and/or piercing member locking mechanism is mediated via a push and twist motion of the first vessel in relation the second vessel. In one or more embodiments, the unlocking of the piercing member and/or piercing member locking mechanism is mediated via a push and twist motion of the first and second vessels in relation to each other. In one or more embodiments, the unlocking of the piercing member and/or piercing member locking mechanism is mediated via a push and twist motion of the first and/or second vessel towards the decontamination device. In one or more embodiments, the unlocking of the piercing member and/or piercing member locking mechanism is mediated via a push and twist motion of the decontamination device towards the first and/or second vessel. In one or more embodiments, the unlocking of the piercing member and/or piercing member locking mechanism is mediated via a push and twist motion of the decontamination device and first and/or second vessel towards each other.

In one or more embodiments, the unlocking of the piercing member and/or piercing member locking mechanism is mediated via a twist and push motion of the second vessel in relation to the first vessel. In one or more embodiments, the unlocking of the piercing member and/or piercing member locking mechanism is mediated via a twist and push motion of the first vessel in relation the second vessel. In one or more embodiments, the unlocking of the piercing member and/or piercing member locking mechanism is mediated via a twist and push motion of the first and second vessels in relation to each other. In one or more embodiments, the unlocking of the piercing member and/or piercing member locking mechanism is mediated via a twist and push motion of the first and/or second vessel towards the decontamination device. In one or more embodiments, the unlocking of the piercing member and/or piercing member locking mechanism is mediated via a twist and push motion of the decontamination device towards the first and/or second vessel. In one or more embodiments, the unlocking of the piercing member and/or piercing member locking mechanism is mediated via a twist and push motion of the decontamination device and first and/or second vessel towards each other.

The present invention further provides a method for establishing a fluidic communication between vessels, the method comprising:

providing a first vessel having a piercing member;

providing a second vessel; and

engaging/coupling the first and second vessels, wherein at about the time of engagement of the vessels, the piercing member pierces through a seal/cover of the first and optionally the second vessel, thereby establishing a fluidic communication between the first and second vessels.

The present invention further provides a method for establishing a fluidic communication between vessels, the method comprising:

providing a first vessel having a piercing member and a piercing member lock mechanism, the piercing member lock mechanism configured to lock the piercing member in a retracted position;

providing a second vessel; and

engaging/coupling the first and second vessels, wherein at about the time of engagement of the vessels, the piercing member locking mechanism is unlocked, allowing the piercing member to pierce through a seal/cover of the first and optionally the second vessel, thereby establishing a fluidic communication between the first and second vessels.

The present invention further provides a method for establishing a fluidic communication between vessels, the method comprising:

providing a first vessel having a piercing member and a piercing member lock mechanism, the piercing member lock mechanism configured to lock the piercing member in a retracted position;

providing a second vessel having a piercing member unlock mechanism, the piercing member unlocking mechanism configured to unlock the piercing member of the first vessel;

engaging/coupling the first and second vessels, wherein at about the time of engagement of the vessels, the piercing member locking mechanism is unlocked, allowing the piercing member to pierce through a seal/cover of the first and optionally the second vessel, and thereby establish a fluidic communication between the first and second vessels.

In one or more embodiments, the herein disclosed devices and systems include a vessel with a plunger and a plunger lock mechanism that can lock the plunger, preventing premature transfer of the content from the vessel. Various forms of the plunger lock mechanism are herein contemplated. Non-limiting examples of the plunger locking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall. The plunger lock mechanism may be moveable between a locked position and an unlocked position. Optionally, the plunger lock position includes extension of the lock mechanism into a hole/dent/socket/channel. Optionally, in the plunger lock position, the lock mechanism is extended into the hole/dent/socket/channel and in the unlock position, the lock mechanism is retracted.

In one or more embodiments, the herein disclosed devices and systems include a plunger unlock mechanism that can unlock the lock mechanism of the plunger. In one or more embodiments, the plunger unlock mechanism unlocks the lock mechanism at about the time of or following an engagement between a first vessel and a second vessel. Various forms of the unlock mechanism are herein contemplated. Non-limiting examples of the unlocking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, and a slot.

In one or more embodiments, the present invention provides a vessel having a moveable wall locking mechanism. In one or more embodiments, the present invention provides a vessel having a moveable wall and a moveable wall locking mechanism. In one or more embodiments, the present invention provides a vessel having a moveable wall unlocking mechanism. In one or more embodiments, the moveable wall is a plunger.

In a further aspect, the present invention provides a method for establishing fluid communication between vessels, the method comprising:

providing a first vessel having a plunger and a plunger locking mechanism;

providing a second vessel having a plunger unlocking mechanism;

connecting the first vessel to the second vessel; and

pushing a plunger of the first vessels towards the second vessel to thereby establishing a fluid passageway between the vessels, wherein the plunger locking mechanism configured to lock the plunger thus preventing the plunger from moving and expelling a substance within the first vessel prior to the engagement between the first vessel and the second vessel, wherein the plunger unlocking mechanism configured to unlock the plunger locking mechanism of the first vessel; wherein upon the connection between the plunger unlocking mechanism and the plunger locking mechanism, the plunger locking mechanism is unlocked by the plunger unlocking mechanism thus allowing the plunger to move and expel the contents of the first vessel into the second vessel.

In one or more embodiments, the unlocking of the plunger locking mechanism is mediated via a pushing motion of the second vessel towards the first vessel. In one or more embodiments, the unlocking of the plunger locking mechanism is mediated via a pushing motion of the first vessel towards the second vessel. In one or more embodiments, the unlocking of the plunger locking mechanism is mediated via a pushing motion of the first and second vessels towards each other.

In one or more embodiments, the unlocking of the plunger locking mechanism is mediated via a twisting motion of the first vessel in relation to the second vessel. In one or more embodiments, the unlocking of the plunger locking mechanism is mediated via a twisting motion of the second vessel in relation to the first vessel. In one or more embodiments, the unlocking of the plunger locking mechanism is mediated via a twisting motion of the first and second vessels in relation to each other.

In one or more embodiments, the unlocking of the plunger locking mechanism is mediated via a turning motion of the first vessel in relation to the second vessel. In one or more embodiments, the unlocking of the plunger locking mechanism is mediated via a turning motion of the second vessel in relation to the first vessel. In one or more embodiments, the unlocking of the plunger locking mechanism is mediated via a turning motion of the first and second vessels in relation to each other.

In one or more embodiments, the unlocking of the plunger locking mechanism is mediated via a push and twist motion of the second vessel in relation to the first vessel. In one or more embodiments, the unlocking of the plunger locking mechanism is mediated via a push and twist motion of the first vessel in relation the second vessel. In one or more embodiments, the unlocking of the plunger locking mechanism is mediated via a push and twist motion of the first and second vessels in relation to each other.

In one or more embodiments, the unlocking of the plunger locking mechanism is mediated via a twist and push motion of the second vessel in relation to the first vessel. In one or more embodiments, the unlocking of the plunger locking mechanism is mediated via a twist and push motion of the first vessel in relation the second vessel. In one or more embodiments, the unlocking of the plunger locking mechanism is mediated via a twist and push motion of the first and second vessels in relation to each other.

In one or more embodiments, at least one of a push and twist motions moves the plunger locking mechanism from a locked position where the plunger is unable to move to an unlocked position where the plunger is can move forward.

In a further aspect, the present invention provides a method for establishing a fluid communication between vessels, the method comprises

providing a first vessel having a plunger lock mechanism, the plunger lock mechanism configured to lock a plunger within the vessel thus preventing premature movement thereof;

providing a second vessel; and

providing a decontamination device having a first and second ports for connection to the first and second vessels, the decontamination device comprising a wiper/wiping member disposed therein the device and configured to remove contaminants from a surface of at least one of the first and second vessels, the decontamination device further comprises a plunger unlock mechanism configured to unlock a plunger lock mechanism of the first vessel,

connecting the first vessel to the decontamination device;

wherein upon the engagement between the first vessel and the decontamination device, the plunger unlock mechanism of the decontamination device unlocks the plunger lock mechanism of the first vessel, thus allowing for the movement of the plunger of the first vessel to expel the contents of the vessel into the decontamination device and thereby establish a fluid communication between the first vessel and the second vessel.

In one or more embodiments, the unlocking of the plunger lock mechanism of the first vessel occurs due to a pushing motion of the decontamination device towards the first vessel. In one or more embodiments, the unlocking of the plunger lock mechanism of the vessel occurs due to a pushing motion of the vessel towards the decontamination device. In one or more embodiments, the unlocking of the plunger lock mechanism of the vessel occurs due to a pushing motion of the vessel and the decontamination device towards each other.

In one or more embodiments, the unlocking of the plunger lock mechanism of the vessel occurs due to a twisting/turning motion of the decontamination device relative to the vessel. In one or more embodiments, the unlocking of the plunger lock mechanism of the vessel occurs due to a twisting/turning motion of the vessel relative to the decontamination device. In one or more embodiments, the unlocking of the plunger lock mechanism of the vessel occurs due to a twisting/turning motion of the vessel and the decontamination device relative to each other.

In one or more embodiments, the unlocking of the plunger lock mechanism of the vessel occurs due to a push and twist motion of the decontamination device relative to the vessel. In one or more embodiments, the unlocking of the plunger lock mechanism of the vessel occurs due to a push and twist motion of the vessel relative to the decontamination device. In one or more embodiments, the unlocking of the plunger lock mechanism of the vessel occurs due to a push and twist motion of the vessel and the decontamination device towards each other.

In one or more embodiments, the unlocking of the plunger lock mechanism of the vessel occurs due to a twist and push motion of the decontamination device relative to the vessel. In one or more embodiments, the unlocking of the plunger lock mechanism of the vessel occurs due to a twist and push motion of the vessel relative to the decontamination device. In one or more embodiments, the unlocking of the plunger lock mechanism of the vessel occurs due to a twist and push motion of the vessel and the decontamination device towards each other.

In one or more embodiments, the herein disclosed devices and systems include a vessel with one or more valves for further controlling the fluid passageway between the vessels. Optionally, the one or more valves include a valve lock mechanism that can lock the valve, preventing premature transfer of the content from the vessel. Optionally, a second vessel or a decontamination device includes a valve unlock mechanism for unlocking the valve/valve lock mechanism.

Various forms of the valve locking mechanism are contemplated and applicable, including without limitation a hook, pin, retractable pin, locking key, latch, knob, lock, dent, hole, channel, bangle, slot, or a combination thereof. Various forms of the valve unlocking mechanism are contemplated and applicable, including without limitation a hook, pin, retractable pin, locking key, latch, knob, lock, dent, hole, channel, bangle, slot.

Various positions for the valve and/or valve lock mechanism and/or valve unlock mechanism are contemplated, such as within the vessel, within a tip or port of the vessel, on a surface of an aperture/tip/port of the vessel, on internal surfaces of side walls of the vessel. on a cavity of the vessel, extending through a wall of the vessel.

In one or more embodiments, the valve locking mechanism engages/couples with the valve unlocking mechanism at about the time of engagement/coupling of the first vessel and the second vessel.

Various motions can be applicable for opening the valve lock mechanism. In one or more embodiments, the unlocking of the valve locking mechanism is mediated via a motion selected from a pushing motion, a twisting motion, a turning motion, a push and twist motion, a twist and push motion or a combination thereof. The motion may be of: the second vessel towards the first vessel, the first vessel towards the second vessel, the first and second vessels towards each other, the decontamination device towards the second vessel, the decontamination device towards the first vessel, the second vessel towards the decontamination device, the first vessel towards the decontamination device, the first and/or second vessels towards the decontamination device.

In one or more embodiments, the unlocking of the valve locking mechanism is mediated via a pushing motion of the first vessel in relation to the second vessel. In one or more embodiments, the unlocking of the valve locking mechanism is mediated via a pushing motion of the second vessel in relation to the first vessel. In one or more embodiments, the unlocking of the valve locking mechanism is mediated via a pushing motion of the first and second vessels in relation to each other.

In one or more embodiments, the unlocking of the valve locking mechanism is mediated via a twisting motion of the first vessel in relation to the second vessel. In one or more embodiments, the unlocking of the valve locking mechanism is mediated via a twisting motion of the second vessel in relation to the first vessel. In one or more embodiments, the unlocking of the valve locking mechanism is mediated via a twisting motion of the first and second vessels in relation to each other.

In one or more embodiments, the unlocking of the valve locking mechanism is mediated via a turning motion of the first vessel in relation to the second vessel. In one or more embodiments, the unlocking of the valve locking mechanism is mediated via a turning motion of the second vessel in relation to the first vessel. In one or more embodiments, the unlocking of the valve locking mechanism is mediated via a turning motion of the first and second vessels in relation to each other.

In one or more embodiments, the unlocking of the valve locking mechanism is mediated via a push and twist motion of the second vessel in relation to the first vessel. In one or more embodiments, the unlocking of the valve locking mechanism is mediated via a push and twist motion of the first vessel in relation the second vessel. In one or more embodiments, the unlocking of the valve locking mechanism is mediated via a push and twist motion of the first and second vessels in relation to each other.

In one or more embodiments, the unlocking of the valve locking mechanism is mediated via a twist and push motion of the second vessel in relation to the first vessel. In one or more embodiments, the unlocking of the valve locking mechanism is mediated via a twist and push motion of the first vessel in relation the second vessel. In one or more embodiments, the unlocking of the valve locking mechanism is mediated via a twist and push motion of the first and second vessels in relation to each other.

Optionally, the valve locking mechanism is configured to be unlocked by a pushing motion of the first vessel towards the second vessel. Alternatively, the valve locking mechanism is configured to be unlocked by a pushing motion of the second vessel relative to the first vessel. Alternatively, the valve locking mechanism is configured to be unlocked by a pushing motion of the first and second vessels towards each other.

In one or more embodiments, at least one of a push and twist motions moves the valve locking mechanism from a locked position where the valve is unable to move to an unlocked position where the valve can move forward.

The present invention further provides a method for establishing a fluidic communication between vessels, the method comprising:

providing a first vessel having a valve;

providing a second vessel; and

engaging/coupling the first and second vessels, wherein at about the time of engagement of the vessels, the valve opens, allowing the valve to open and establishment of a fluidic communication between the first and second vessels.

The present invention further provides a method for establishing a fluidic communication between vessels, the method comprising:

providing a first vessel having a valve and a valve lock mechanism, the valve lock mechanism configured to lock the valve in a closed position;

providing a second vessel; and

engaging/coupling the first and second vessels, wherein at about the time of engagement of the vessels, the valve locking mechanism is unlocked, allowing the valve to open and establishment of a fluidic communication between the first and second vessels.

The present invention further provides a method for establishing a fluidic communication between vessels, the method comprising:

providing a first vessel having a valve and a valve lock mechanism, the valve lock mechanism configured to lock the valve in a closed position;

providing a second vessel having a valve unlock mechanism, the valve unlocking mechanism configured to unlock the valve of the first vessel;

engaging/coupling the first and second vessels, wherein at about the time of engagement of the vessels, the valve locking mechanism is unlocked, allowing the valve to open and establishment of a fluidic communication between the first and second vessels.

In one embodiment, the present invention further provides devices, systems and methods configured to decontaminate one or more surfaces of a vessel(s). In one or more embodiments, the devices and systems of the invention allow a substantially decontaminated fluid transfer between the one or more vessel(s). The engagement and/or fluid transfer between the one or more vessels may accordingly be utilized via the decontamination devices which decontaminate the vessel(s) and also allow the fluid transfer. A valve and/or piercing member may be disposed on the decontamination device, and one or both engaging vessels. A locking mechanism and/or an unlocking mechanism of one or more of a valve, a piercing member and a plunger may be disposed on the decontamination device, and one or both engaging vessels.

Optionally, the decontamination device includes an actuator, disposed through a wall of the device, for manipulating the movement of the wiping member, and thereby decontaminating the surface of the first and/or second vessels. In one or more embodiments, the actuator is a handle. In one or more embodiments, the actuator is a tab.

Optionally, the locking mechanism of a valve and/or a plunger and/or the piercing member may be disposed on the vessel and/or the unlocking mechanism of a valve and/or a plunger and/or the piercing member on the decontamination. Various positions of the locking/unlocking mechanism on the vessel and/or decontamination device are herein contemplated. The locking/unlocking mechanism may be disposed, without limitation, on a vessel's/device's body, port, aperture, wall, plunger and/or compartment. The locking mechanism may be disposed in an external and/or internal position of the vessel/device. Optionally, the decontamination device includes one or more compartments. Optionally, the decontamination device includes in a first compartment thereof a connecting structure/port for receiving a vessel. The connecting structure/port may mediate a connection with a vessel, via an engagement mechanism. Various forms of connecting structures/ports are herein contemplated. In one or more embodiments, the connecting structure/port extends downwardly from a top inner wall of a first compartment. In one or more embodiments, the connecting structure/port includes extension bodies (e.g., an elongated arm), clamping members, a threaded structure, minimized built-in dent structures, elongated extensions, and a circular body.

Various form of first and/or second vessels are contemplated. For example, the vessel may be a cartridge. In one or more embodiments, the cartridge includes protective side walls. In one or more embodiments, the moveable wall is a plunger. In one or more embodiments, the moveable wall is made of a rubber material. In one or more embodiments, the moveable wall is made of a plastic material. In one or more embodiments, the moveable wall is made of an elastomeric material. In one or more embodiments, at least one of the first and second vessels is a bag. In one or more embodiments, at least one of the first and second vessels is a bottle. In one or more embodiments, the first vessel is a bag and the second vessel is a syringe. In one or more embodiments, the first vessel is a syringe and the second vessel is a bag. In one or more embodiments, the first vessel is a bottle and the second vessel is a syringe. In one or more embodiments, the second vessel is a cartridge having a plunger.

Various types, combinations and numbers of plunger and/or valve and/or piercing member locking mechanisms may be provided in each of the herein disclosed systems, to ensure the locking action. For example, a combination of two locking mechanisms or more may be provided in each device/system/vessel.

Various types, combinations and numbers of plunger and/or valve and/or piercing member unlocking mechanisms may be provided in each of the herein disclosed systems, to ensure the unlocking action. For example, a combination of two unlocking mechanisms or more may be provided in each device/system/vessel. In one or more embodiments, the locking mechanism is unlocked by the unlocking mechanism at the time of engagement of the first and second vessels. In one or more embodiments, the locking mechanism is unlocked by the unlocking mechanism after the engagement of the first and second vessels. In one or more embodiments, the locking and/or unlocking mechanism is disposed through a wall of the vessel. In one or more embodiments, the locking and/or unlocking mechanism is disposed on an interior wall of the vessel. In one or more embodiments, the locking and/or unlocking mechanism is disposed on an exterior wall of the vessel.

In one or more embodiments, the valve and/or plunger and/or piercing member unlock mechanism is operable to unlock a lock mechanism by displacing/removing a lock mechanism (e.g., a pin) from a channel/slot. In one or more embodiments, the valve and/or plunger and/or piercing member unlock mechanism is operable to unlock a lock mechanism by displacing/removing a lock mechanism (e.g., a stopper) from a dent. In one or more embodiments, the valve and/or plunger and/or piercing member unlock mechanism is operable to unlock a lock mechanism by displacing/removing a stopper from a protrusion of a valve/piercing member/plunger. In one or more embodiments, the valve and/or plunger and/or piercing member unlock mechanism is operable to unlock a lock mechanism by displacing/removing a latch from a valve and/or a plunger and/pre a piercing member.

In one or more embodiments, a portion of the valve and/or plunger and/or piercing member unlocking mechanism that engages/couples with the valve and/or plunger and/or piercing member locking mechanism is disposed in a wall of the vessel, inside the vessel, on an inner surface of the vessel or on an outer surface of the vessel.

In one or more embodiments, a portion of the valve and/or plunger and/or piercing member locking mechanism that engages/couples with the valve and/or plunger and/or piercing member unlocking mechanism is disposed in a wall of the vessel, inside the vessel, on an inner surface of the vessel or on an outer surface of the vessel.

In one or more embodiments, any of the valve locking mechanisms, piercing member locking mechanisms, and/or plunger locking mechanisms is selected from a stopper, a latch, a dent, a hole, a channel, a slot, a pin, a retention member, a fastener, and combinations thereof.

In one or more embodiments, any of the valve unlocking mechanisms, piercing member unlocking mechanisms, and/or plunger unlocking mechanisms is selected from a stopper, a latch, a dent, a hole, a channel, a slot, a pin, a retention member, a fastener, and combinations thereof.

Optionally, the valve, piercing member, and/or plunger includes a spring, optionally disposed in a wall of the vessel, for allowing an energy/force that will open/close the valve and/or piercing member and/or plunger.

Optionally, the valve locking mechanisms, piercing member locking mechanisms, and/or plunger locking mechanisms includes a spring, optionally disposed in a wall of the vessel, for allowing an energy/force that will open the valve and/or piercing member and/or plunger.

The decontamination device of the invention includes a housing having side walls, a top wall and a bottom wall, and a wiping member disposed within the housing. The device configured to be coupled to at least two vessels and includes applicable connecting structures/ports or openings for receiving the vessels. The connecting structures/ports/openings may optionally be formed by vertical walls extending from the top and/or bottom walls, forming an inner lumen via which medical substances may be transferred following engagement between, and decontamination of the vessel surfaces. Other port configurations include ports/openings without any walls. The ports may be flush, or surface mounted to the housing. Optionally, a locking and/or unlocking may be disposed in a port of the device, for example, in an internal position within vertical walls of the port.

The wiping member is configured to decontaminate a vessel's surface, optionally by a movement of the wiping member across the housing and surface of the vessel, or by movement of the surface of the vessel within the housing. The wiping member may be moveable or may be static. Movement of the wiping member across the housing has the effect of pushing contaminants across the surface of at least one of the vessels, pushing the contaminants away from the location of where a fluidic communication will be established between vessels (optionally between a vessel and a decontamination device), quarantining the contaminants in a chamber of the decontamination device which is separate from the location of the establishment of the fluidic communication, and/or removing all air particles and dust/dirt from the location of where the fluidic communication will be established between vessels (optionally a vessel and a decontamination device.

In an embodiment of the invention, the wiping member is operable to move across and wipe off any contaminates present on the one or more surfaces of the vessels via an actuator extending through an opening of the housing. The actuator is configured to manipulate movement of the wiping member across the housing. In one or more embodiments, the wiping member is operable to move across and wipe off any contaminants present on the one or more surfaces of the vessels via an actuator extending through an opening in the side wall of the housing. Exemplary actuators include, without limitation, a handle, a tab, a button, a touch button, a lever, a gear, a spring, a mechanical actuator, an electric actuator, an air pressure actuator, and combinations thereof.

In one or more embodiments, the wiping member is operable to move across and wipe off any contaminates present on the one or more surfaces of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member.

Optionally, the wiping member is static within the housing and the decontamination of a vessel's surface is effective via movement of the vessel within the housing and via the wiping member. In accordance with this embodiment, the system optionally comprises a sliding mechanism positioned on at least one of the first vessel and the second vessel, and/or within the housing, the sliding mechanism configured to allow traveling there along of at least one of the first vessel and the second vessel, such that at least one of the first vessel and the second vessel is configured to move from a first position to a second position. The wiping member is configured to remove contaminants from a surface of at least one of the first vessel and the second vessel at about the time of movement of the medical vessels from the first position to the second position. In one or more embodiments, the medical vessel or medical vessel surface slides along the housing and passes through the wiping member under great friction, or under a friction sufficient to wipe off any contaminants from the surface of the vessel, such as dirt, bacteria or ambient air particles.

Various types of sliding mechanisms are contemplated as long as they can be connected or engage a vessel and afford traveling there along and allow decontamination of a surface of the vessel. The sliding mechanism may include a rail. The rail may be optionally made from a plastic or polymetric material, but a metallic material or glass material is also applicable. In one embodiment, the rail is manufactured from a plastic material. In one or more embodiments, the plastic material is rigid. In one or more embodiments the vessel surface is positioned on a surface of the rail. In one or more embodiments, the vessel surface is positioned between the rails of the rail. In one or more embodiments, the vessel surface is positioned between a portion of the rails. In one or more embodiments, the rail may be a circumferential rail. In one or more embodiments, the sliding mechanism may include a hinge/pivot mechanism. In one or more embodiments, the sliding mechanism provides for the travelling a vessel between a plurality of positions, such as from a second position to a third position, from a third position to a fourth position, and from a fourth position to a fifth position. In one or more embodiments, the sliding mechanism allows only unidirectional sliding. Alternatively, the sliding mechanism allows for the vessel to retract/return to a previous position.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria. In one or more embodiments, the vessel's surface slides underneath the wiping member under great friction, or under a friction sufficient to wipe off any contaminants from the surface, such as dirt or bacteria.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof.

In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings and/or ports of the decontamination device are sealed and/or covered thus preventing the sterilizing substance from escaping from inside the housing of the decontamination device. The seals and/or covers maintain the sterility of the decontamination device until about the time of use of the decontamination device. In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

Various sizes and shapes of the housing are applicable. For example, the housing may have an external most length, width, and/or height of less than about 3 inches. In one or more embodiments, the housing may have an external most length, width, and/or height of less than about 25 millimeters (mm). For example, less than about 20 mm, less than about 15 mm, or less than about 10 mm. The housing internal most length, width, and/or height may be less than about 10 mm. For example, less than about 4 mm, less than about 3 mm, or less than about 2 mm. The housing may include one or more compartments which may optionally be separated or divided by the wiping member or by any other means. In one or more embodiments, the housing includes two compartments, wherein a first compartment is kept sterile and a second compartment is configured to receive a vessel which may be contaminated. In one or more embodiments, the sterile compartment can receive a vessel following decontamination of the surface of the vessel. In one or more embodiments, the housing includes two compartments wherein a first includes a capping mechanism and the second is configured to receive a vessel. In one or more embodiments, the housing includes three, four, or five compartments. The vessel may move from a first position in which a vessel surface is located within the first compartment to a second position in which the vessel surface is located in the second compartment. Optionally, the housing includes a third compartment and the vessel surface is moveable from the second position to a third position. Optionally, the housing includes a fourth compartment, or a fifth compartment and the vessel surface is moveable from a third position to a fourth position and/or a fifth position.

In one or more embodiments, the housing is conveniently light presenting a weight of no more than about 100 grams (gr). For example, less than about 75 gr, less than about 50 mm gr, less than about 25 gr, less than about 10 gr, or less than about 5 gr. In one or more embodiments, the housing may be transparent allowing visualization of its interior, or opaque.

In one or more embodiments, the housing may be a closed chamber which forms a tight, optionally, airtight connection between the vessels. In one or more embodiments, when the ports or openings are connected to vessels or when the ports are covered by a cover or a seal, the housing may form an interior which is hermetically sealed/isolated from surrounding/environmental air. In one or more embodiments, the cover or the seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing is already connected to one or more medical vessels. In accordance with this embodiment, the one or more medical vessels may be integrally manufactured or assembled with the housing and provided or distributed to consumers as such. In accordance with this embodiment, the system may be configured to allow a connection/locking/engagement to additional one or more vessels.

In one or more embodiments, the system includes two or more housings each containing a wiping member. Optionally, the housings are disposed one above the other, providing a top housing and a bottom housing. In accordance with this embodiment, a wiping member disposed on the top housing is configured to wipe a surface of a vessel connected to the top housing and a wiping member disposed on the bottom housing is configured to wipe a surface of a vessel connected to the bottom housing.

In one or more embodiments, the system includes two or more ports which are disposed one above the other, or which are longitudinally aligned with respect to each other. In accordance with this embodiment, the vessels connected to such ports are positioned aligned allowing a direct and aligned fluid passageway between the vessels. In one or more embodiments, the system includes two or more ports which are shifted from each other. Yet, in accordance with this embodiment, a fluid passageway is formed between vessels which is twisted or curved. In yet one or more embodiments, the housing contains a plurality of ports configured for connecting a plurality of vessels to the housing. The system or device is configured so that actuating the wiping member allows wiping the surfaces of all vessels connected to the housing in one action.

In one or more embodiments, the housing has an engagement mechanism configured or adapted to connect the at least one vessel to the housing. In one or more embodiments, the engagement mechanism may be reversible, allowing to detach the engagement between a vessel and a decontamination device. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In one or more embodiments, the herein disclosed invention allows transferring medical substances in a contaminant-free, or in a substantially contaminant-free manner.

The term “lock mechanism is interchangeable with the term “locking mechanism”.

The term “unlock mechanism is interchangeable with the term “unlocking mechanism”.

In one or more embodiments, the herein disclosed invention affords an engagement of vessels in a contaminant-free, or in a substantially contaminant-free manner. In one or more embodiments, the herein disclosed invention affords an engagement of vessels in an air-free, or in a substantially air-free manner.

In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner.

In one or more embodiments, the herein disclosed invention, allows to substantially decrease the chances to introduce contaminants within a medical substance, when preparing or administering medical substances for administration to patients.

As used herein the term “substances” refers to various types of materials that should be kept sterile. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the terms a “pharmaceutical substance”, “a beneficial substance” and “a medicament”.

As used herein the term “contaminant-free” is interchangeable with the term “sterile”, “disinfected”, and “decontaminated”. The term refers to substances that are free or substantially free of ambient air particles and/or pathogens and/or microorganisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. In an embodiment of the invention, the vessel is utilized for containing or housing a medical substance. Various types of vessels are contemplated. The vessel may be selected, without limitation, from a vial, a syringe, a bag, a chamber, a bottle, a container, a cartridge and the alike. In an embodiment of the invention, the term vessel further encompasses elements that can be used to connect between vessels. In accordance with this embodiment, the vessel may be selected, without limitation, from a connector, a connector having a plurality of openings, a syringe, an infusion line, a tubing, an infusion line, a spike, a syringe, a filter, a port and a manifold. In one or more embodiments, the first container is a bag. In one or more embodiments, the herein system include a first vessel and a second vessel. In one or more embodiments, the first vessel is a cartridge configured to fill the second vessel. In accordance with this embodiment, the cartridge includes a mechanism for allowing the fluid transfer. Suitable fluid transfer mechanisms may include, without limitation a plunger, and a moveable wall. Optionally, the cartridge may have various forms, such as a syringe, a vial, a bottle, a bag, etc. In one or more embodiments, the first vessel is a bottle. In one or more embodiments, the first vessel is a syringe and the second vessel is a bag, or vice versa. In one or more embodiments, the first vessel is a syringe and the second vessel is a bottle, or vice versa. In one or more embodiments, the first vessel is a cartridge with a moveable wall and the second vessel is a bag. In one or more embodiments, the first vessel is a cartridge with a plunger and the second vessel is a bag. In one or more embodiments, the first vessel is a cartridge with a moveable wall and the second vessel is a bottle. In one or more embodiments, the first vessel is a cartridge with a plunger and the second vessel is a bottle. In one or more embodiments, at least one of the first and second containers is a bag. In one or more embodiments, at least one of the first and second vessels is a bottle. In one or more embodiments, the vessel is a cartridge having a plunger and protective side walls.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between two containers, between two bottles, between a syringe and a bag, between a syringe and a container, between a syringe and a vial, between a connector and a bag, between a container and a bag, between a vial and a bag, between a container and an infusion line or between a syringe and a connector. Optionally, one vessel is configured to connect a plurality of vessels. For example, one vessel can connect two, or three, or four or five, or six or more vessels. Optionally, a second vessel can connect a plurality of first vessels. Optionally, the second vessel includes a plurality of cavities, each configured to accommodate and/or connect a vessel. In an embodiment of the invention, the herein disclosed devices and systems are disposable. In an embodiment of the invention, the herein disclosed devices and systems are non-disposable.

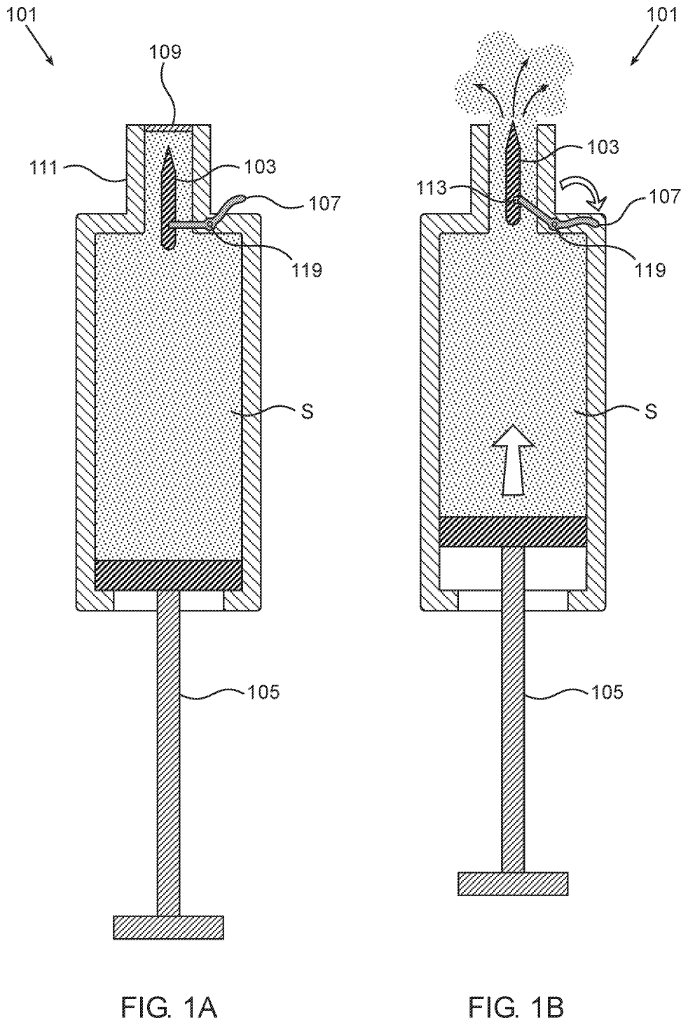

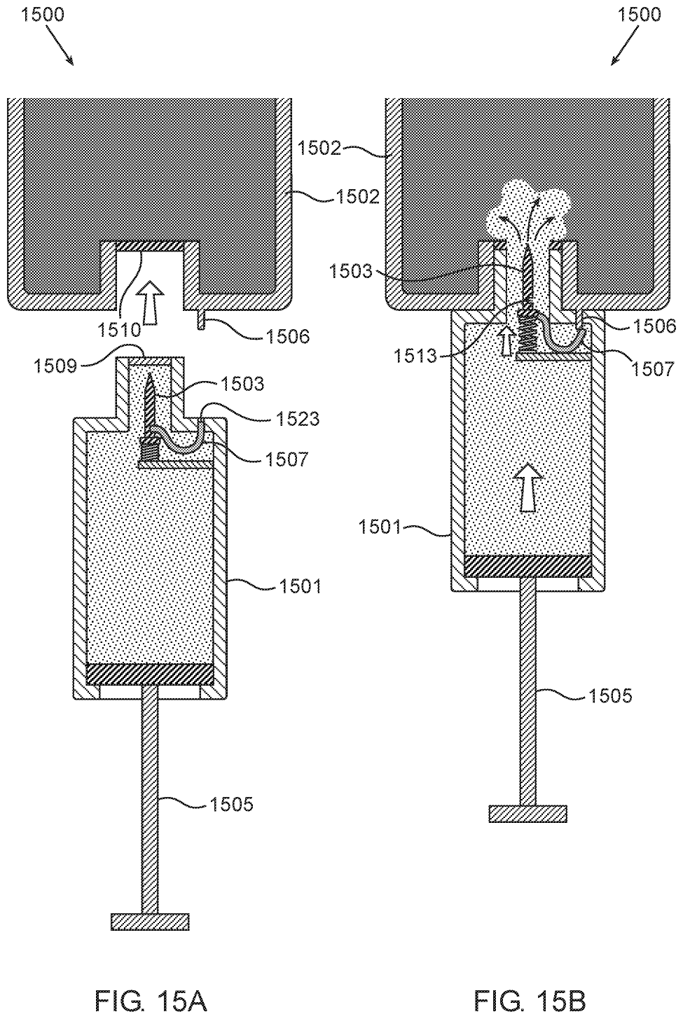

Referring now to the drawings, FIGS. 1A-1B illustrate a syringe 101 having a piercing member 103 disposed inside the syringe 101. Specifically, piercing member 103 is disposed within a lumen of tip 111, optionally in the middle of tip 111. Having the piercing member 103 disposed inside the syringe 101 protects it from ambient air contaminants and keeps it sterile. The piercing member 103 further includes a piercing member lock mechanism 107 connected at a first internal end thereof to the piercing member 103 and configured to lock it in an initial retracted position wherein the piercing member 103 is locked and unable to move. A second external end of the piercing member lock mechanism 107 allows maneuvering thereof to elevate the piercing member 103 and thereby pierce seal 109. Piercing member 103 may include a dent 113 adapted in size and shape to allow first internal portion/end of the lock mechanism 107 to enter (be positioned inside) the dent 113, thus locking the piercing member 103 to prevent premature elevation thereof and piercing through seal 109 of the syringe. Second external portion of the lock mechanism 107 extends and protrudes through a wall of the syringe 101 to the exterior environment. Optionally, the exterior portion of the lock mechanism is configured to unlock the lock mechanism 107 upon engagement of the syringe 101 with another vessel. Alternatively, and/or additionally, the exterior portion of the lock mechanism 107 is configured/adapted to engage/couple with a piercing member unlocking mechanism of another vessel (shown for example in FIGS. 15A-15B). Piercing member 103 is optionally moveable/rotatable via a hinge 119 which allows movement thereof about hinge 119.

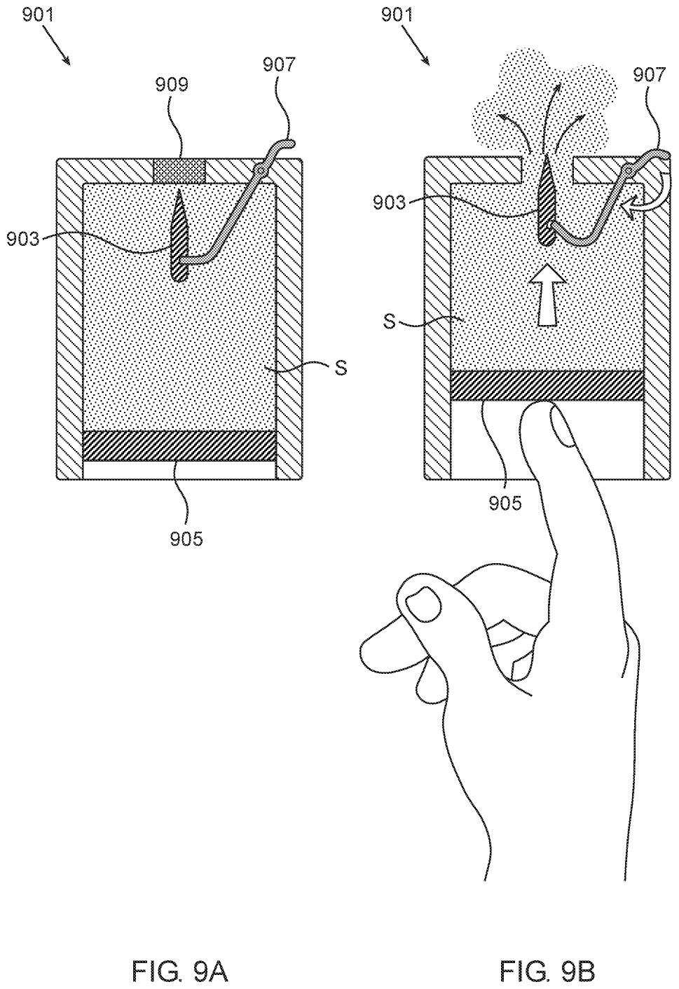

The syringe 101 has a plunger 105 which when pushed upwardly by a user expels fluid substance “S” out of the syringe 101. Optionally, the plunger 105 is configured to expel substance “S” at about the time of, at the time of and/or after piercing of seal 109 by piercing member 103. At FIG. 1A, piercing member 103 is shown in a retracted locked configuration wherein seal 109 is intact, thus fluid substance “S” is maintained inside the syringe 101 and cannot flow therefrom. FIG. 1B shows syringe 101 when the piercing member 103 is in an advanced position wherein the lock mechanism 107 releases holding from piercing member 103 allowing it to pierce seal 109 and substance “S” to flow out of the syringe 101. The abovesaid mechanism of a medical vessel having a piercing member disposed within the vessel and a lock mechanism that prevents the premature seal piercing is applicable to other forms of vessels, e.g., a medical vessel/bottle/container/bag/vial/cartridge. An exemplary alternative vessel is shown in FIGS. 9A-9B which illustrate a cartridge 901 having a similar piercing member 903 and piercing member lock mechanism 907. FIG. 9A illustrates the cartridge 901 wherein the piercing member 903 in the locked position and FIG. 9B illustrates the cartridge 901 wherein the piercing member 903 in the unlocked advanced position and capable of piercing through seal 909. Upon a user (or another vessel) pushing downwardly the lock mechanism 907 the piercing member 903 is lifted upwardly and can pierce through seal 909.

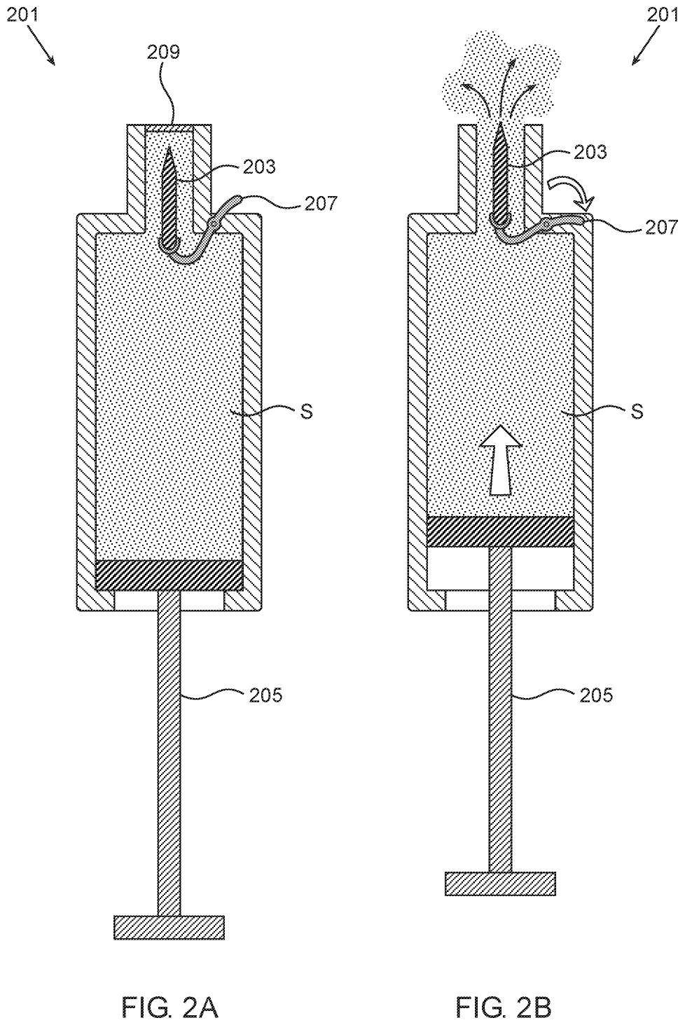

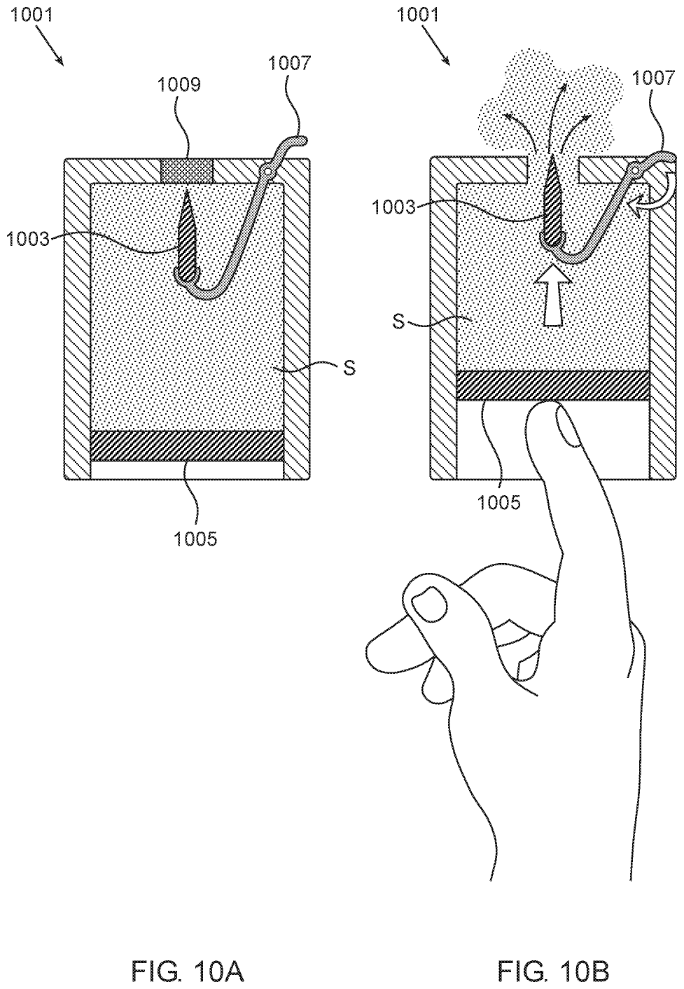

FIGS. 2A-2B illustrate yet another syringe 201 which includes a plunger 205, a piercing member 203 and a piercing member lock mechanism 207. The piercing member lock mechanism 207 has a form of a latch which grasps an inner portion, optionally a protruding portion, of the piercing member 203 and thereby locks the piercing member 203 maintaining it in an initial locked position. The piercing member lock mechanism 207 prevents premature spillage/expulsion of any substance/medicament that may be contained inside the syringe 201 prior to the syringe engaging/coupling with another container/medical device. Such latch mechanism and piercing member may be incorporated in alternative forms of containers/vessels such as cartridge 1001 shown in FIGS. 10A-10B. FIGS. 10A-10B illustrate cartridge 1001 having a piercing member 1003 and piercing member lock mechanism in the form of a latch 1007. FIG. 10A illustrates the cartridge 1001 wherein the piercing member 1003 in the locked position and FIG. 10B illustrates the cartridge 1001 wherein the piercing member 1003 in the unlocked advanced position and capable of piercing through seal 1009.

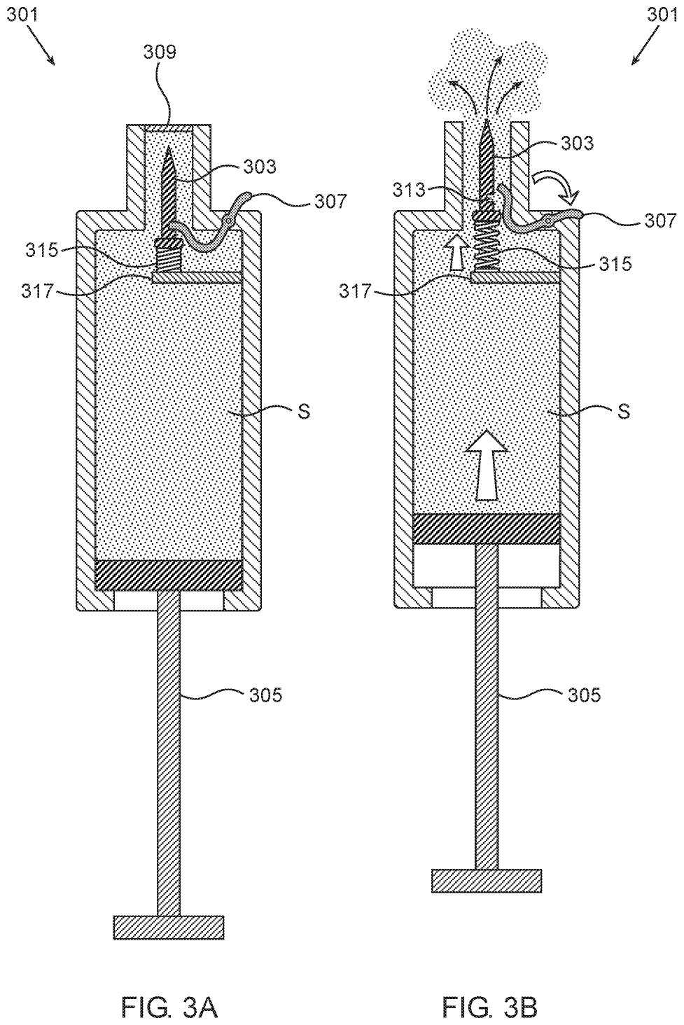

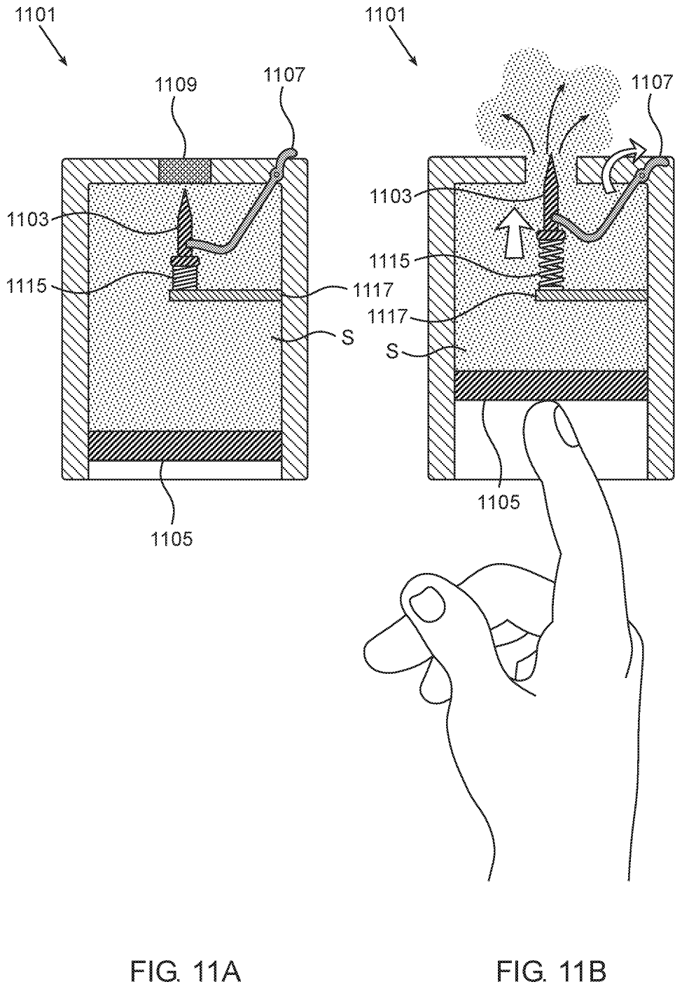

FIGS. 3A-3B illustrate yet another syringe 301 which includes a plunger 305, a piercing member 303 and a piercing member lock mechanism 307. An inner end of the piercing member lock mechanism 307 is connected to piercing member 303 via dent 313. The piercing member 303 is connected to syringe 301 via support base 317 and spring 315. The support base 317 supports and stabilizes the piercing member 303 in syringe 301. When unlocking lock mechanism 307, e.g., by pushing and/or twisting downwardly the outer portion/end of lock mechanism 307, spring 315 can be extended, thereby pushing upwardly/lifting piercing member 303 such to pierce seal 309. FIG. 3A shows the piercing member 303 in a locked position and unable to move forward and pierce through seal 309. FIG. 3B shows the piercing member lock mechanism 307 in an unlocked position where the inner portion of the lock mechanism 307 displaced from dent 313, allowing the piercing member 303 to move forward and pierce through seal 309. Similar supported piercing member may be incorporated in alternative forms of containers/vessels such as cartridge 1101 shown in FIGS. 11A-11B. FIGS. 11A-11B illustrate cartridge 1101 having a piercing member 1103 and piercing member lock mechanism 1107 connected to piercing member 1103. FIG. 11A illustrates the cartridge 1101 wherein the piercing member 1103 in the locked position and FIG. 11B illustrates the cartridge 1101 wherein the piercing member 1103 in the unlocked position and capable of piercing through seal 1109. The lock mechanism 1107 can assist in elevating piercing member 1103 and thereby pierce through seal 1109.

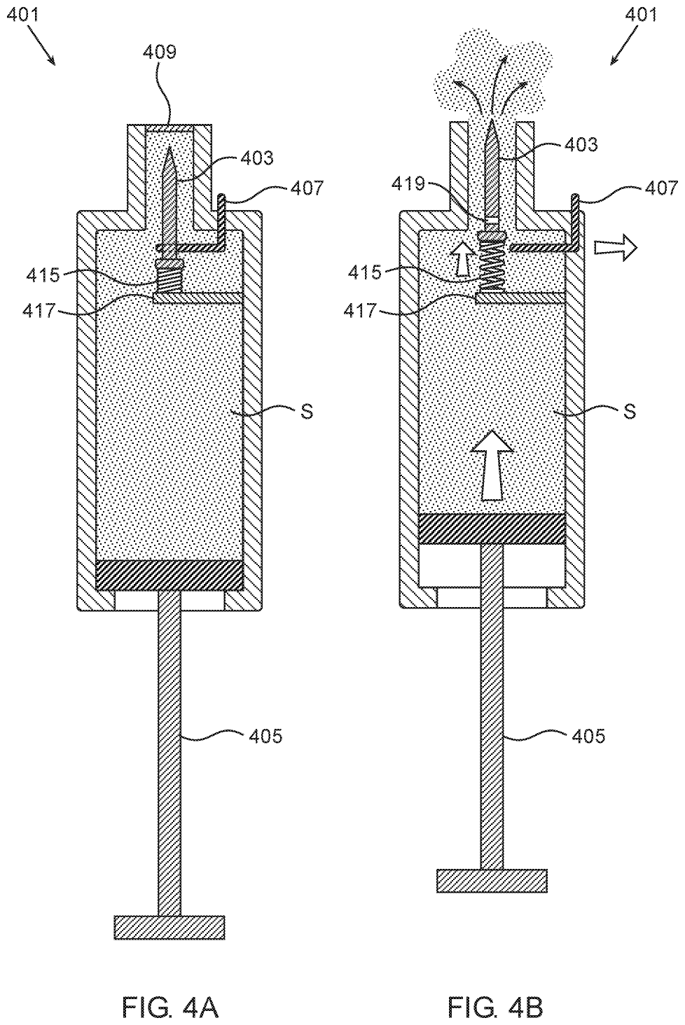

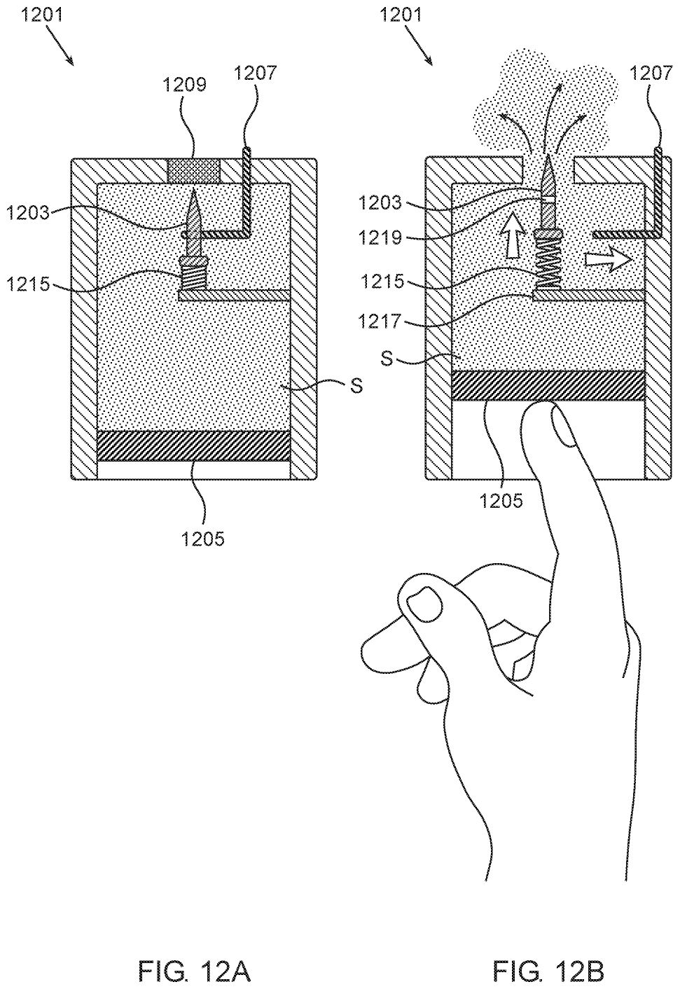

FIGS. 4A-4B illustrate syringe 401 having piercing member 403 with a slot/channel 419 via which an inner portion of piercing member lock mechanism 407 is disposed thus preventing the piercing member 403 from moving forward and piercing through seal 409. FIG. 4A shows the piercing member 403, and piercing member lock mechanism 407 in a locked position wherein the piercing member 403 unable to move due to positioning of inner portion of lock mechanism 407 in slot/channel 419. FIG. 4B illustrates lock mechanism 407 in an unlocked position when displaced from slot 419. The lock mechanism is unlocked by pulling out (displacing) lock mechanism 407 from slot/channel 419. Similar piercing member with a slot may be incorporated in alternative forms of containers/vessels such as cartridge 1201 shown in FIGS. 12A-12B. FIGS. 12A-12B illustrate cartridge 1201 having a piercing member 1203 and piercing member lock mechanism 1207 connected to piercing member 1203 via slot 1219. FIG. 12A illustrates the cartridge 1201 wherein the piercing member 1203 in the locked position and FIG. 12B illustrates the cartridge 1201 wherein the piercing member 1203 in the unlocked position and capable of piercing through seal 1209.

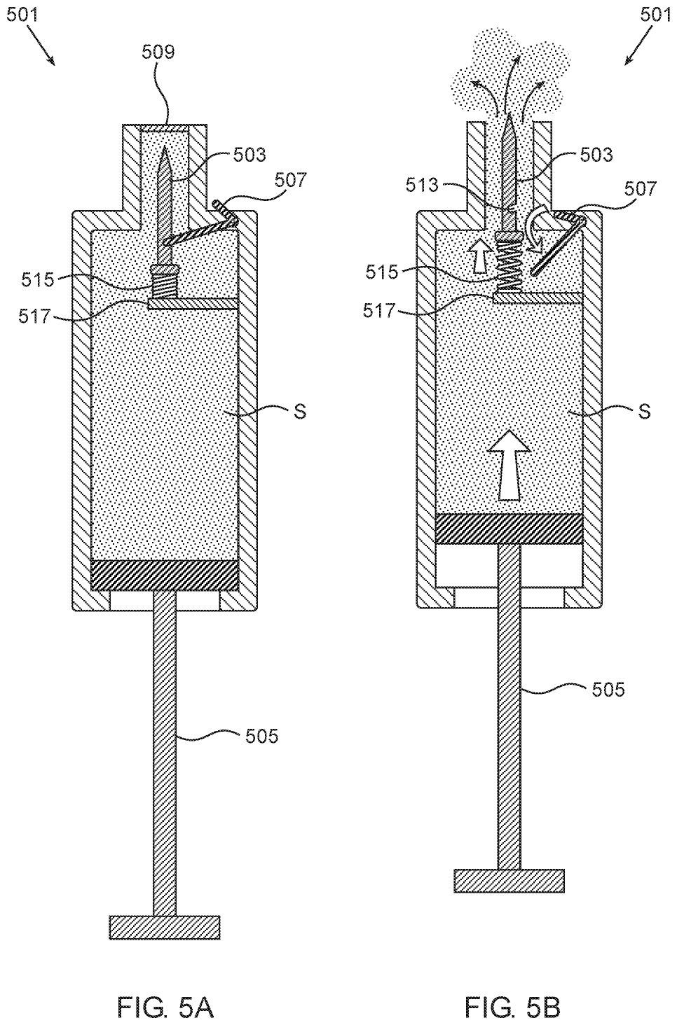

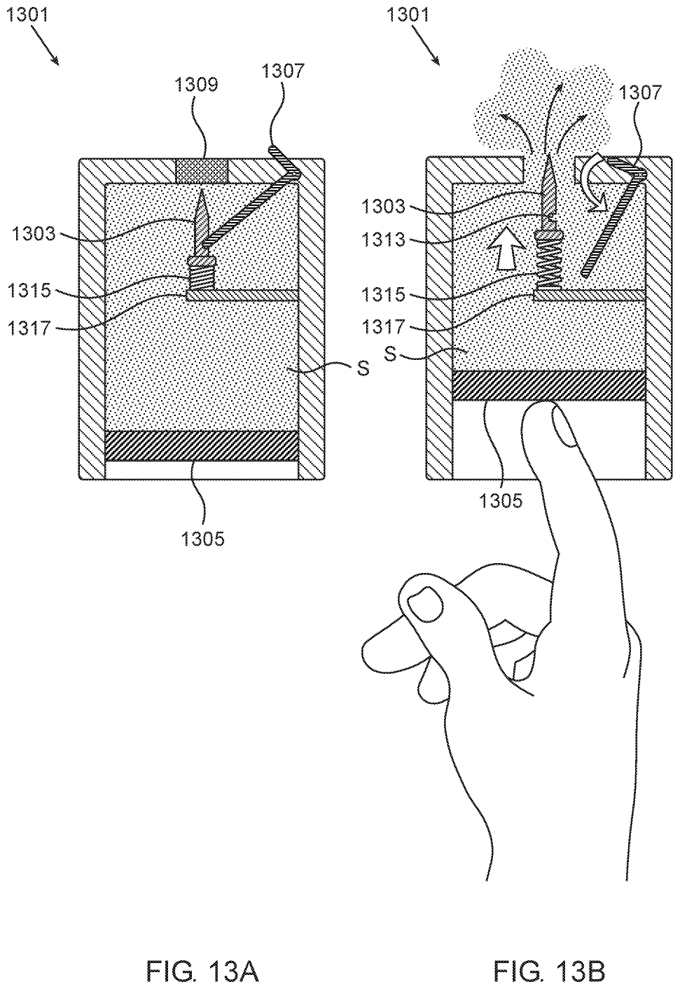

FIGS. 5A-5B illustrate yet another syringe 501 which includes a piercing member 503 and a piercing member lock mechanism 507 wherein an inner end thereof is connected to piercing member 503 via dent 513. The piercing member 503 is connected to syringe 501 via support base 517 and spring 515. Here, the unlocking of lock mechanism 507 includes pushing and/or twisting inwardly, to the left of syringe 501, the exterior portion of lock mechanism 507, thereby extending spring 515 and lifting piercing member 503 such to pierce seal 509. FIG. 5A shows the piercing member 503 in a locked position and unable to move forward and pierce through seal 509. FIG. 5B shows the piercing member lock mechanism 507 in an unlocked position where the inner portion of the lock mechanism 507 displaced from dent 513, allowing the piercing member 503 to move forward and pierce through seal 509. Similar supported piercing member may be incorporated in alternative forms of containers/vessels such as cartridge 1301 shown in FIGS. 13A-13B. FIGS. 13A-13B illustrate cartridge 1301 having a piercing member 1303 and piercing member lock mechanism 1307 connected to piercing member 1303 via dent 1313. FIG. 13A illustrates the cartridge 1301 wherein the piercing member 1303 in the locked position and FIG. 13B illustrates the cartridge 1301 wherein the piercing member 1303 in the unlocked position and capable of piercing through seal 1309.

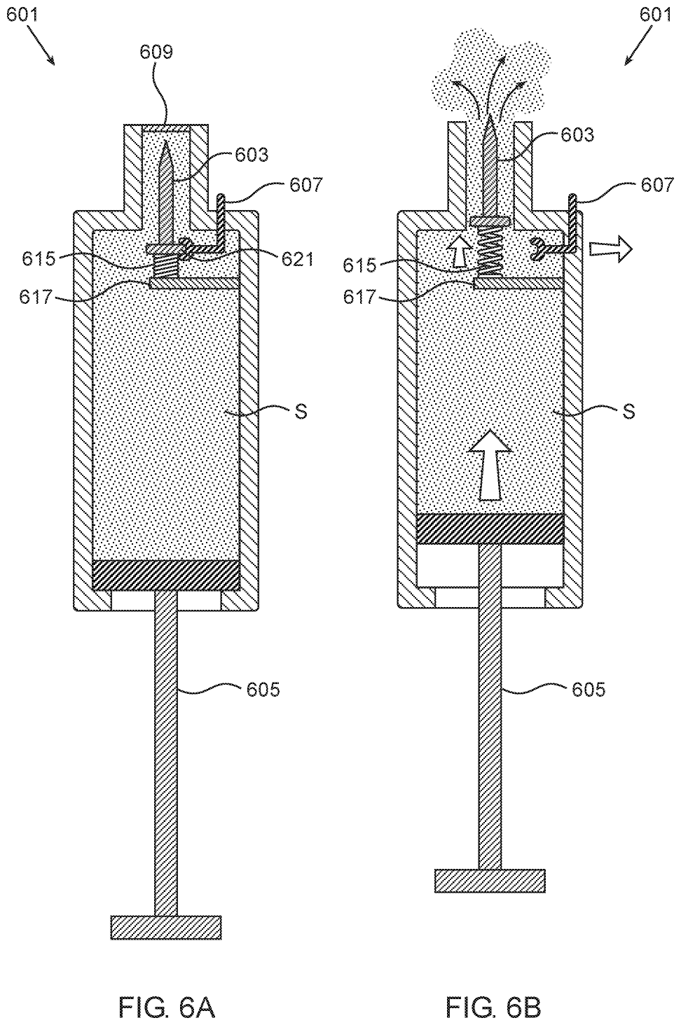

FIGS. 6A-6B illustrate syringe 601 having a piercing member 603 locked by piercing member lock mechanism having a form of latch 607. The latch 607 holds a protrusion 621 extending from a side facet of piercing member 603. Latch 607 which grasps protrusion 621 locks the piercing member 603, maintaining it in an initial locked position. The piercing member lock mechanism 607 prevents premature spillage/expulsion of any substance/medicament that may be contained inside the syringe 601. FIG. 6A illustrates the syringe 601 wherein the piercing member 603 in the locked withdrawn/retracted position. The dislocation/displacement of latch 607 allows an extension of spring 615 and lifting of piercing member 603. Cartridge 1401 includes a similar piercing member 1403, and piercing member lock mechanism 1407.

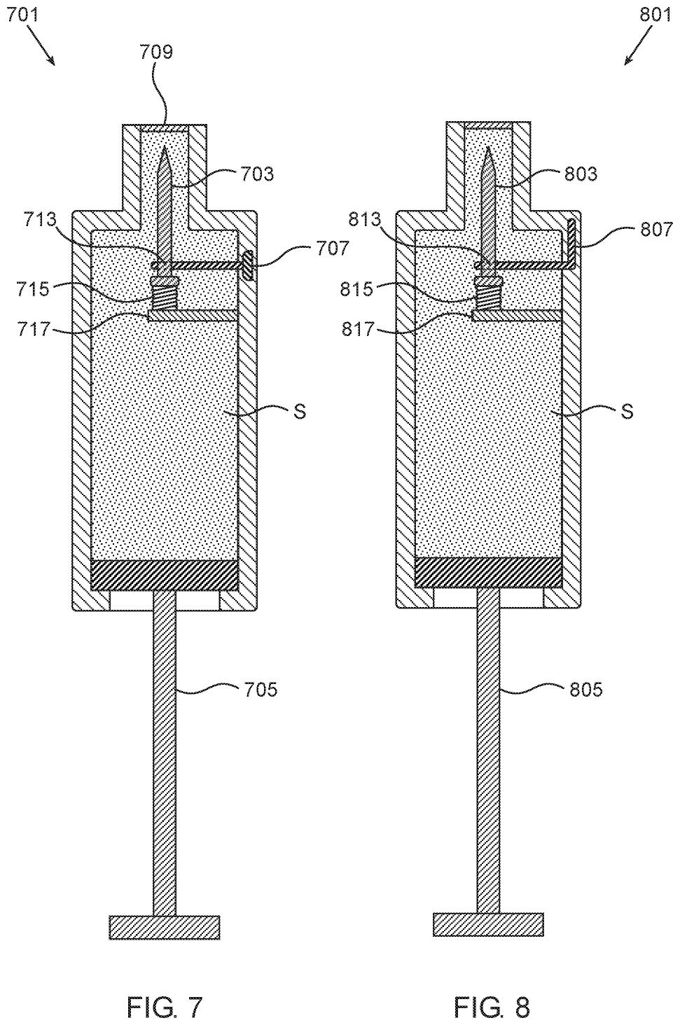

FIGS. 7 and 8 illustrate yet additional syringes 701 and 801 having piercing members 703 and 803, and lock mechanisms thereof 707 and 807, respectively. Internal ends of the lock mechanisms 707 and 807 disposed in slots 713 and 813, of syringes 701 and 801 respectively. Both lock mechanisms 707 and 807 include opposing ends which are slotted within the side walls of syringes 701 and 801, respectively. Having lock mechanisms 707 and 807 opposing ends disposed inside the side walls of the syringes is very important as it prevents the accidental/premature unlocking of the lock mechanisms 707 and 807 and thus accidental/premature movement of piercing members 703 and 803 prior to syringe 701 and 801 engagement to another vessel. In FIG. 7 the lock mechanism 707 has a T shaped structure and in FIG. 8 the lock mechanism 807 has an L-shaped structure.

FIGS. 15A-15B illustrate a system 1500 which includes a first vessel, here syringe 1501, and a second vessel 1502. First vessel 1501 and second vessel 1502 are configured to be coupled to each other and allow fluid transfer therebetween. First vessel 1501 includes a piercing member 1503 and a piercing member lock mechanism 1507. Second vessel 1502 includes a piercing member unlock mechanism in the form of a tenon 1506 extending externally from a bottom surface of the vessel 1502 and which geometrically fits and can be inserted into a dedicated socket 1523 in vessel 1501. The first vessel 1501 and/or second vessel 1502 may accommodate a beneficial substance which may be a medicament, nutritional substance, a diagnostic substance, a diluent, a sterile fluid, etc. The piercing member unlock mechanism 1506 is configured to engage with the piercing member lock mechanism 1507 and thereby to unlock the lock mechanism 1507 and allow the movement of piercing member 1503. The engagement between vessel 1501 and vessel 1502 includes insertion of unlock mechanism 1506 into socket 1523 and displacement of lock mechanism 1507 from dent 1513. The displacement of lock mechanism 1507 from attachment with piercing member 1503 will enable unlocking thereof, expansion/release of spring 1515 and movement forward/elevation of piercing member 1503, resulting in puncturing seal 1509 of vessel 1501 and optionally also seal 1510 of vessel 1502. A fluid passageway between vessels 1501 and 1502 may then be established. FIG. 15A shows vessels 1501 and 1502 when spaced apart from each other. FIG. 15B shows first vessel 1501 engaged/coupled to second vessel 1502. The coupling of the syringe to the vessel may be via an engagement mechanism (now shown) which may include a thread, a luer, a luer-lock, a luer-slip, a ratchet teeth mechanism, and/or any other type of engagement mechanism. At about the time, at the time, and/or after the engagement/coupling of the vessels 1501 and 1502, the piercing member unlock mechanism 1506 engages/couples with the piercing member lock mechanism 1507. The engagement/coupling of piercing member unlock mechanism to the piercing member lock mechanism unlocks the piercing member lock mechanism, allowing movement of the piercing member and seal 1509 puncturing, resulting in the establishment of a fluidic communication between the vessels. The unlocking of the lock mechanism 1507 of vessel 1501 may be mediated by a push motion, a twist motion, a push and twist motion, or a twist and push motion of the second vessel's unlock mechanism relative to the first vessel's lock mechanism(s), or vice versa. The unlocking of the piercing member lock mechanism 1507 may include a displacement, as herein shown, but can optionally include removal of the piercing member lock mechanism from the vessel itself.

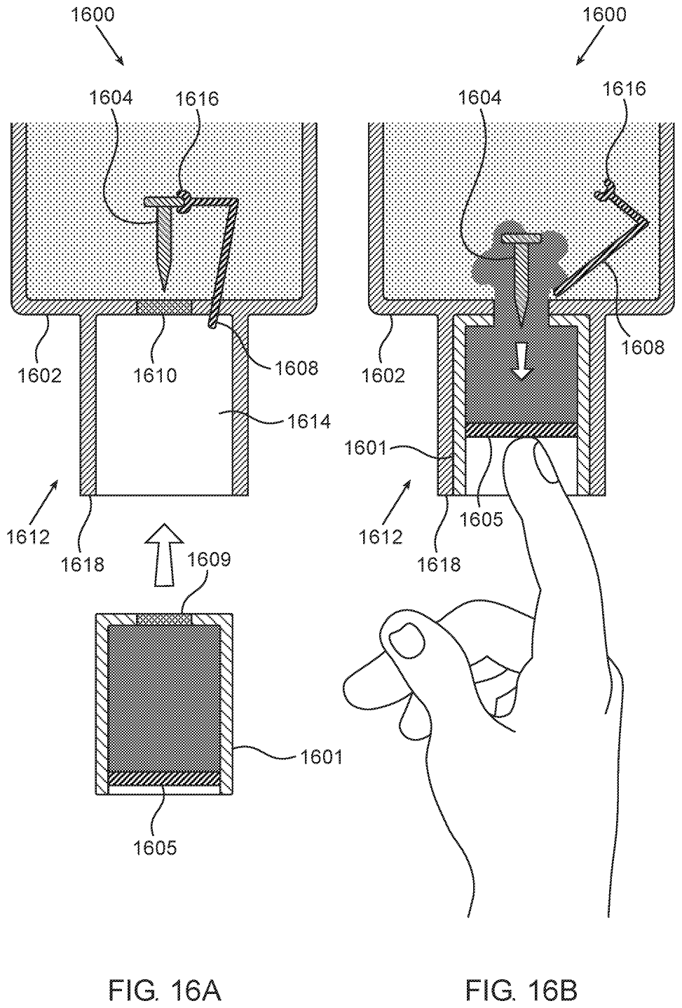

FIGS. 16A-16B illustrate a system 1600 with first vessel in the form of cartridge 1601 (but may be a vial, syringe, etc.) and second vessel 1602. Vessel 1602 may be, but is not limited to, a bag, bottle, and/or container. Here, the second vessel 1602 includes a piercing member 1604 and a piercing member lock mechanism 1608. Second vessel includes a port 1612 with circumferential side walls 1618 and a cavity 1614 for accommodating therein first vessel 1601. Piercing member lock mechanism 1608 includes a first end having a latch like form that holds protrusion 1616 and a second external end that extends out of vessel 1602 into cavity 1614. The engagement between the vessels 1601 and 1602 pushes latch 1608, enabling displacement thereof and unblocking of piercing member 1604. The piercing member can now move forward and puncture seal 1610 and optionally also seal 1609, allowing the establishment of a fluidic passageway between the vessels.

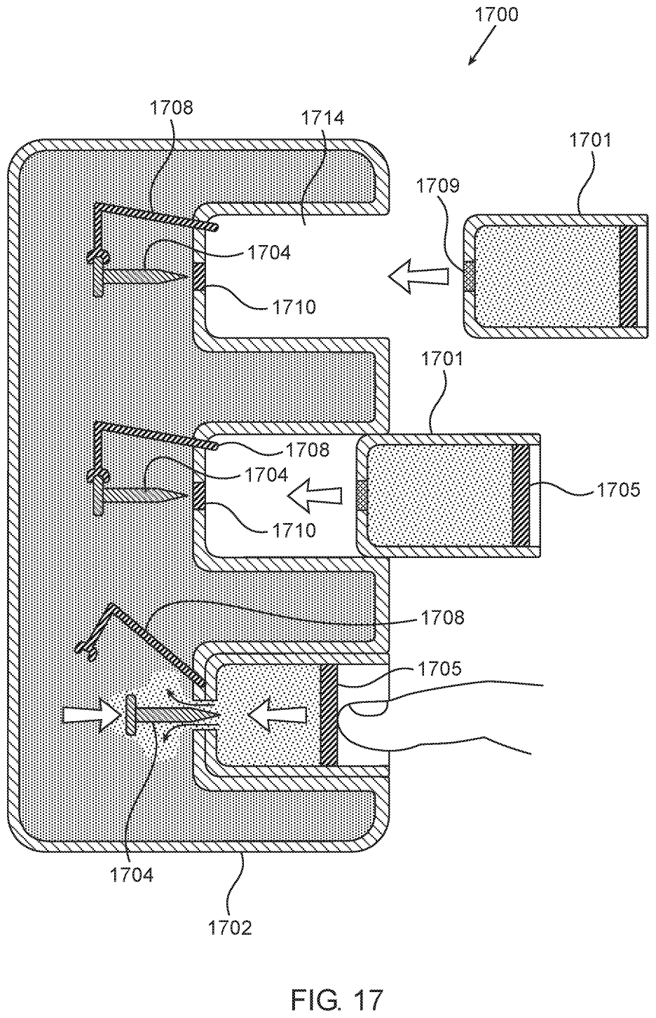

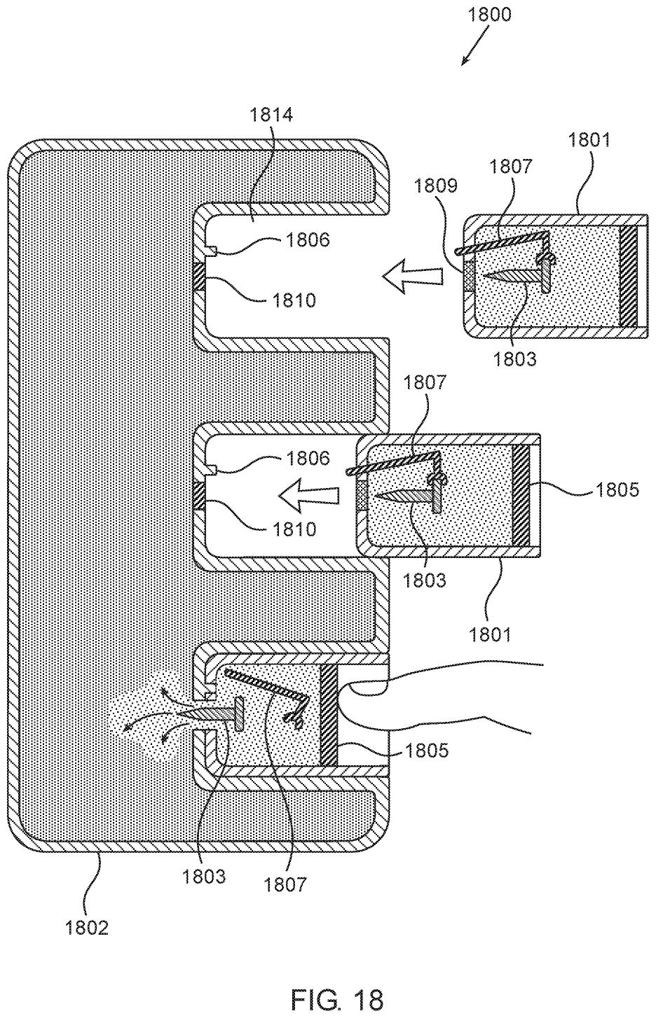

The following FIGS. 17 and 18 illustrate systems 1700 and 1800 with a plurality of first vessels and a second vessel that can be coupled thereto. Said systems constructed such that the plurality of first vessels feed the second vessel, or vice versa with a fluid substance. Various forms of first and/or second vessels are contemplated and applicable for systems 1700 and 1800. Further, those systems may include two, three, four, five or more first vessels that can be engaged with a second vessel. In FIG. 17, a second vessel in the form of bag/bottle 1702 includes a plurality of cavities 1714 for accommodating therein first vessels 1701. First vessels are in the form of cartridges 1701 but alternative vessel types are herein contemplated and applicable, such as a bottle, a vial, a syringe etc. The second vessel 1702 includes piercing members 1704 attached to piercing member lock mechanisms 1708. The piercing members 1704 disposed such to allow puncturing seals 1710 and optionally also of seals 1709 at the time of engagement, or after engagement, between vessels 1701 and vessel 1702.

In System 1800 shown in FIG. 18, piercing members 1803 and piercing member lock mechanisms 1807 are disposed adjacent ports of vessels 1801 to pierce seals 1809. The second vessel 1802 includes piercing member unlock mechanisms in the form of tenons 1806 that are configured to unlock lock mechanisms 1807 at about the time of or at the time of engagement between vessels 1801 and vessel 1802.

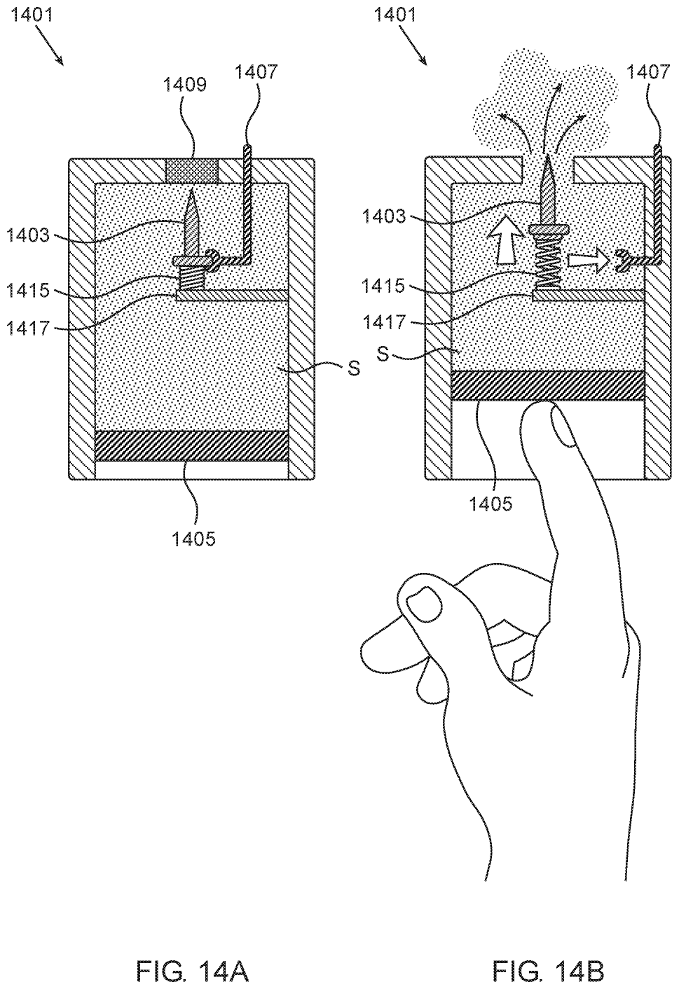

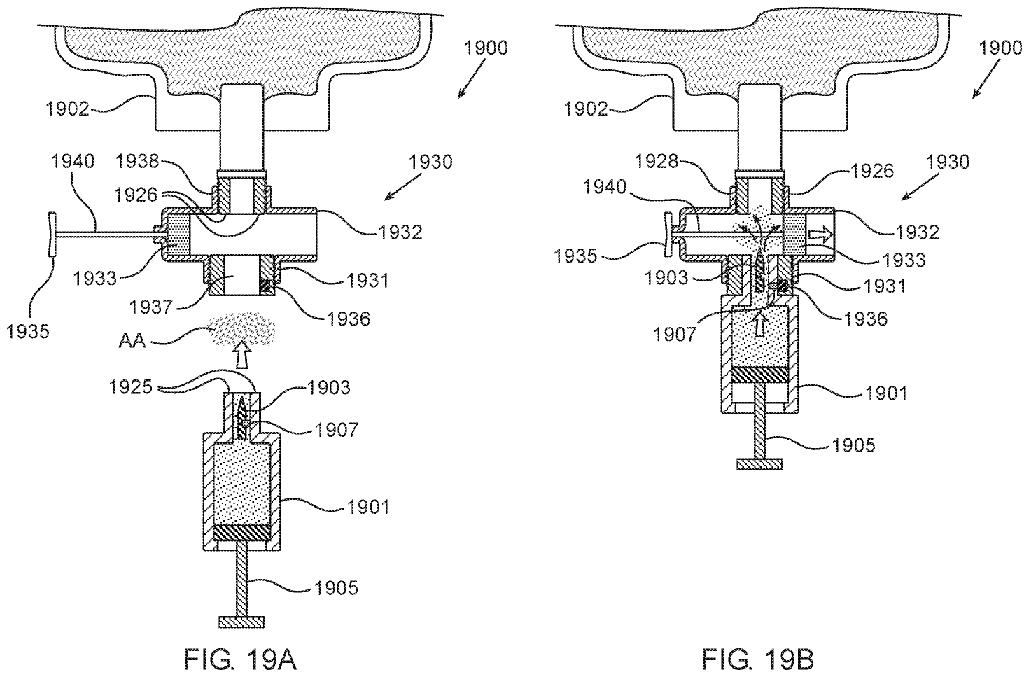

FIGS. 19A-19B illustrate system 1900 for fluid transfer between vessels 1901 and 1902 that includes a decontamination device 1930 having a housing 1932 configured to decontaminate one or more of the surfaces 1925 and/or 1926 of vessels 1901 and 1902, respectively. First vessel 1901 is having a spring-like form and second vessel has a bag-like form, nevertheless alternative forms of first and second vessels are contemplated and applicable. In system 1900, the connection between vessels 1901 and 1902 is via the decontamination device 1930. Decontamination device 1930 includes a first port 1931 which encloses a lumen 1937 for accommodating therein first vessel 1901 or a portion thereof, or a port thereof. Decontamination device 1930 further includes a second port 1938 for accommodating therein second vessel 1902 or a portion thereof, or a port thereof. The decontamination device 1930 has a moveable wiping member 1933 configured to move across the device 1930 and to thereby decontaminate surfaces 1925 and/or 1926. Wiping member 1933 is manipulated externally to move across the device 1930 via a wiping member actuator having a form of an actuator/handle 1935 having a rod 1940. Rod 1940 positioned through a wall of the device 1930 and connects handle 1935 to the wiper 1933. When the handle 1935 is pushed to the right and/or slides across the decontamination device 1930 by a user, the wiper 1933 moves across the decontamination device 1930 and wipes off surfaces 1925 and/or 1926. In system 1900, a piercing member 1903 and a lock mechanism thereof 1907 disposed within a tip of syringe 1901. A piercing member unlocking mechanism 1936 is disposed within port 1931 of decontamination device 1930. The piercing member unlocking mechanism 1936 is shown disposed on an inner surface of a wall of port 1931 of the decontamination device 1930. However, various alternative locations for the piercing member unlocking mechanism 1936 may be applicable. The piercing member unlocking mechanism 1936 may be disposed on a surface of the decontamination device, on an exterior surface of the decontamination device, on an interior surface of the decontamination device, extending through a wall of the decontamination device, located inside a wall of the decontamination device, and/or disposed on/in a port of the decontamination device. The unlock mechanism 1936 includes a magnetic ball which can attract a lock mechanism 1907. The unlock mechanism 1936 may be any of the unlock mechanisms shown in FIGS. 1-18 of this specification. Optionally, vessel(s) 1901 and/or 1902 may be provided when already attached, optionally integrally manufactured with device 1930. At about the time of, at the time of, or following the engagement between first vessel 1901 and decontamination device 1930, piercing member locking mechanism 1907 is unlocked by the unlock mechanism 1936 (FIG. 19B). Surfaces 1925 and/or 1926 can be decontaminated in parallel, or thereafter the engagement by manipulation of actuator/handle 1935 (FIG. 19B). A fluid transfer may then be performed by a user pushing plunger 1905 across decontamination device 1930 (or optionally towards second vessel 1902). FIG. 19A shows the decontamination device 1930 and second vessel 1902 when connected to each other but spaced apart from first vessel 1901. Ambient Air “AA” having contaminants (particles) surrounds between vessel 1901 and decontamination device 1930. FIG. 19B shows the syringe 1901 disposed inside port 1931 of the decontamination device 1930. The syringe 1901 is engaged/coupled to the decontamination device 1930 and a surface 1925 and/or surface 1926 wiped off by wiper 1933 that is actuated by a user by pushing/pulling/sliding the handle/tab 1935 across/into the decontamination device, thus decontaminating surfaces 1925 and/or 1926 of the vessels 1901 and/or 1902. Upon and/or after puncturing a seal by piercing member 1903, a fluidic communication may be established between vessels 1901 and 1902. It is to be noted that any of the lock/unlock mechanisms, and vessels disclosed herein above and in the following figures is applicable to the herein disclosed system 1900. Piercing member 1903 pierces through a surface of the syringe 1901, and optionally through a surface of the decontamination device 1930, after the wiping member/wiper 1933 decontaminates surfaces 1925 and 1926 of the syringe 1901 and bag 1902 respectively. The preferred sequence of establishing a fluidic communication is engagement of syringe 1901 to decontamination device 1930, then movement of wiping member 1933 across the housing of the decontamination device 1930 which decontaminates surfaces 1925 of syringe 1901 and optionally surfaces 1926 of vessel 1902, then unlocking of the piercing member lock mechanism 1907 by piercing unlock mechanism 1936, and finally piercing of surfaces 1925 of syringe 1901 and piercing of surfaces 1926 of vessel 1902 (or decontamination device 1930) by the piercing member 1903. However, in one or more embodiments, unlocking of the syringe's 1901 piercing member lock mechanism 1907 may occur prior to decontamination of vessel surface 1925 and 1926 by wiping member 1933. Alternately, the syringe 1901 may have a piercing member unlock mechanism (not shown) and the decontamination device 1930 may have a piercing member (not shown) and a piercing member lock mechanism (not shown).

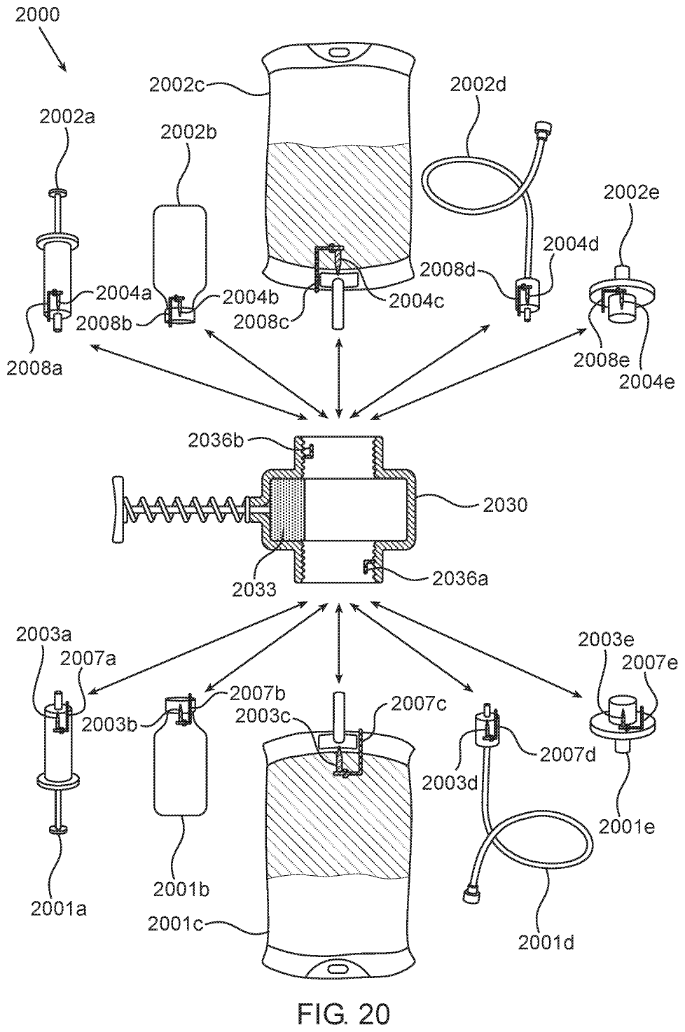

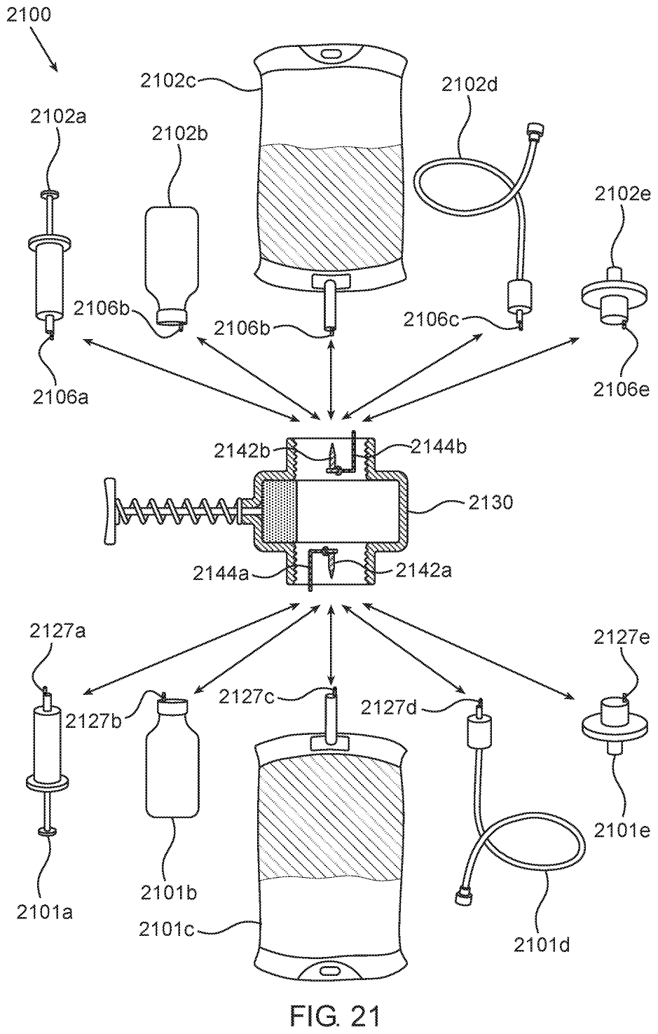

Systems with a decontamination device having a moveable wiping member that can be used as connectors are shown in FIGS. 20-21. The systems 2000-2100 illustrate connectors configured to be coupled to a first and a second vessel and a wiping member that is disposed within the connector and that can wipe off the surface of both vessels. In system 2000 (FIG. 20), the decontamination device 2030 is in the form of a connector that can be coupled to a first and second vessels. Various forms of first vessels are contemplated, such as, syringe 2002a, bottle 2002b, bag 2002c, infusion line 2002d and filter 2002e. Various forms of second vessels are contemplated, such as, syringe 2001a, bottle 2001b, bag 2001c, infusion line 2001d and filter 2001e. The first vessels may include a piercing member 2004 and a piercing member lock mechanism 2008. A piercing member 2003, with a similar lock mechanism 2007 or any other lock mechanism as herein described, may be disposed on/within second vessels. The connector 2030 further includes a piercing member unlocking mechanisms 2036a and 2036b. The wiper 2033 configured to move across the decontamination device 2030 and wipe off a surface of first vessel and/or second vessel upon engagement/coupling thereof to the decontamination device 2030.

In system 2100, (FIG. 21), the decontamination device 2130 is in the form of a connector that can be coupled to a first and second vessels. Various forms of first vessels are contemplated, such as, syringe 2101a, bottle 2101b, bag 2101c, infusion line 2101d and filter 2101e. Various forms of second vessels are contemplated, such as, syringe 2102a, bottle 2102b, bag 2102c, infusion line 2102d and filter 2102e. In system 2100, the connector 2130 includes a piercing members 2142a and 2142b and piercing member locking mechanisms 2144a and 2144b. The first vessels may include a piercing member unlock mechanism 2127a-2127e and the second vessels include a piercing member unlock mechanism 2106a-2106e.

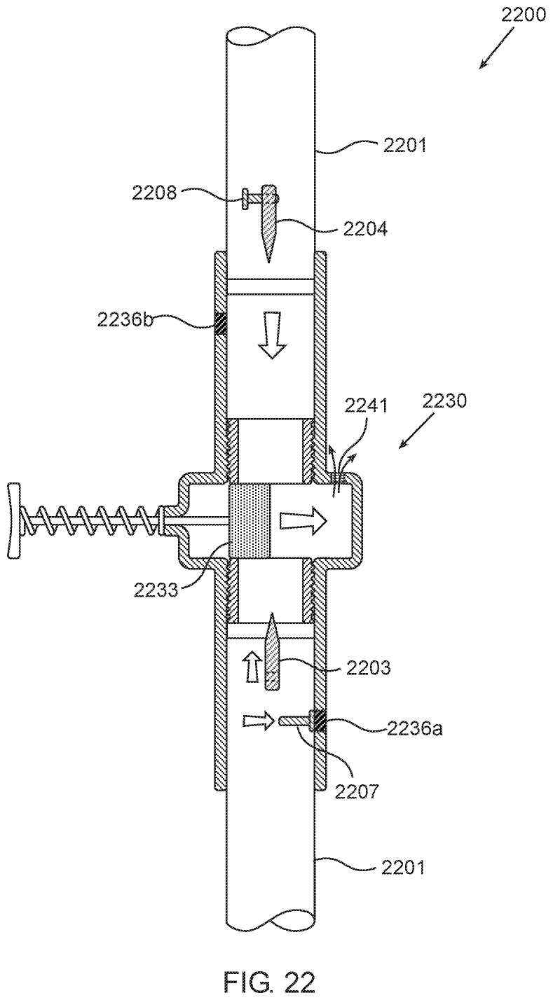

FIG. 22 illustrates yet another exemplary system 2200 which includes a decontamination device 2230 for coupling to vessels 2201 (top) and 2201 (bottom), or ports thereof. Each of the vessels 2201 (top) and 2201 (bottom) includes a piercing member, i.e., piercing members 2204 and 2203, respectively. The piercing members 2204 and 2203 kept locked via piercing member lock mechanisms 2208 and 2207, respectively. Unlock mechanisms 2236b and 2236a are provided to unlock lock mechanisms 2208 and 2207, respectively. Decontamination device 2230 further includes a filter 2244 to allow releasing air from the device when actuating the wiping member 2233.

In system 2300 of FIGS. 23A-23B, the decontamination device 2330 includes a wiping member (not shown) moveable via movement of tab 2345. Tab 2345 is disposed on the exterior of the decontamination device and is connected to the wiper (not shown) disposed on the interior of the device 2330. A first vessel 2301 in the form of a bottle includes a piercing member 2303 which is locked by lock mechanism 2307 in the form of a pin. In FIG. 23A, the vessel 2301 is shown prior to an engagement with decontamination device 2330. Ambient Air “AA” surrounds the vessel 2301. Following the engagement between vessel 2301 and device 2330, the wiping member can be moved by a user moving the tab 2345 from left to right (FIG. 23B). As the tab 2345 slides across the decontamination device 2330, the wiper moves across the interior of the decontamination device 2330 and wipes off a surface of vessel 2301 and/or 2302. A piercing member 2320 may be further disposed in a port or adjacent an aperture of vessel 2302 and/or device 2330. Further the system includes a valve 2324 and a valve actuator 2322 that can be manipulated by a user to further monitor the fluid transfer between the vessels 2301 and 2302. At about the time of vessels engagement, the piercing member 2303, piercing member 2320 and valve 2324 can be operable to allow and establish a fluid communication between vessels 2301 and 2302.

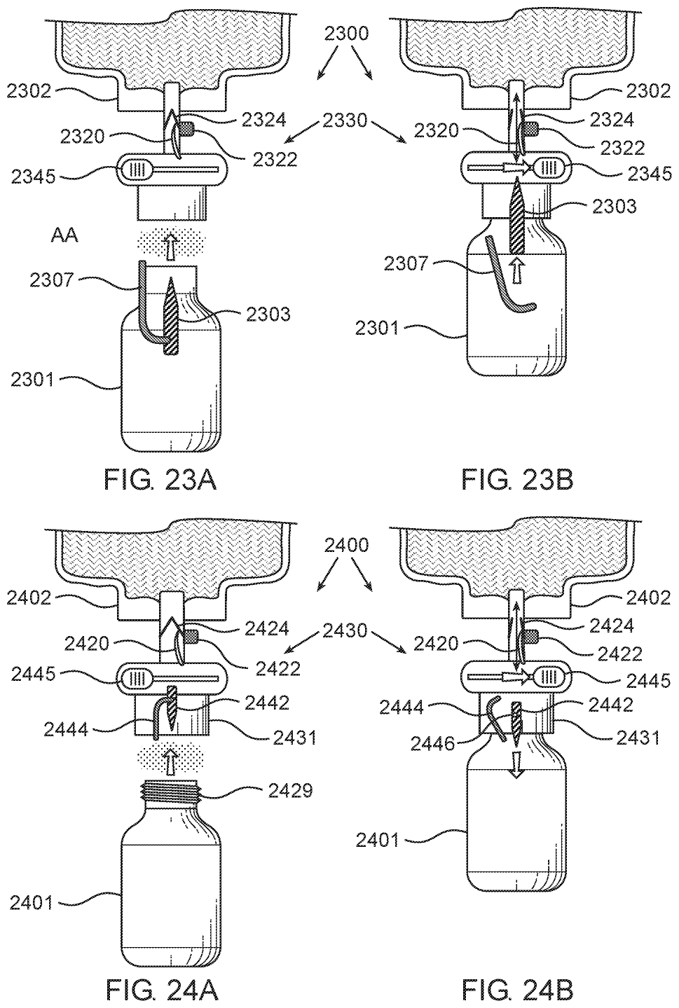

FIGS. 24A-24B illustrate system 2400 which is similar system 2300 but here a piercing member 2442 and a piercing member lock mechanism 2444 disposed within a port 2431 of decontamination device 2430. Piercing member 2442 is configured to pierce through a seal/cover/cap of one or both of decontamination device 2430 and vessel 2401. An engagement mechanism in the form of thread 2429 and a complementary thread in port 2431 (not shown) may be incorporated to system 2400, to allow safe connection between vessel 2401 and device 2430. Optionally, a ratchet teeth mechanism (not shown) may be incorporated into system 2400 to allow safe connection between vessel 2401 and device 2430. The system 2400 further includes a valve 2424 and a valve actuator 2422 which mediates the opening of valve 2424. Valve 2424 may be a one-way valve. Yet a further piercing member 2420 may be disposed in a port or adjacent an aperture of vessel 2402 and/or device 2430. Piercing member lock mechanism 2444 is unlocked by vessel 2401 when vessel 2401 engages/attaches/couples to decontamination device 2430. Engagement/attachment/coupling of vessel 2401 to decontamination device 2430 (as shown in FIG. 24B) displaces/moves piercing member lock mechanism 2444 from dent 2446 of the piercing member 2442 thus unlocking piercing member 2442 and allowing piercing member 2442 to pierce through a surface of vessel 2401.

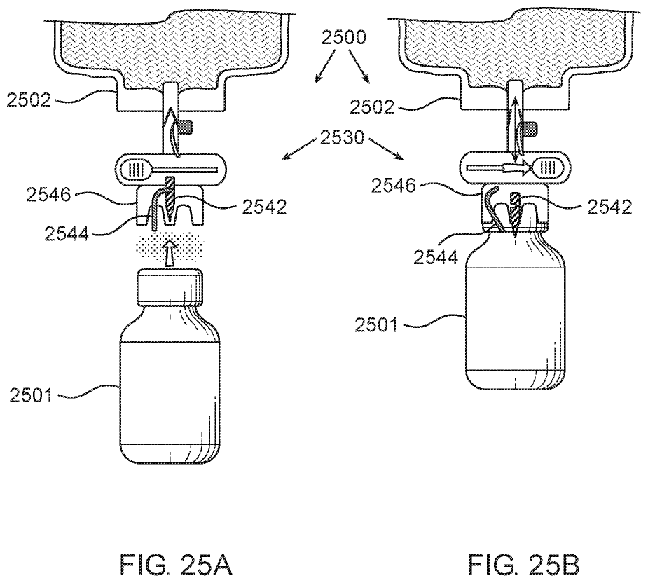

FIGS. 25A-25B illustrate a system 2500 which is similar to system 2400 but here the engagement mechanism between vessel 2501 and device 2530 includes ratchet teeth 2546 which grasp vessel 2501, optionally grasp a port of vessel 2501.

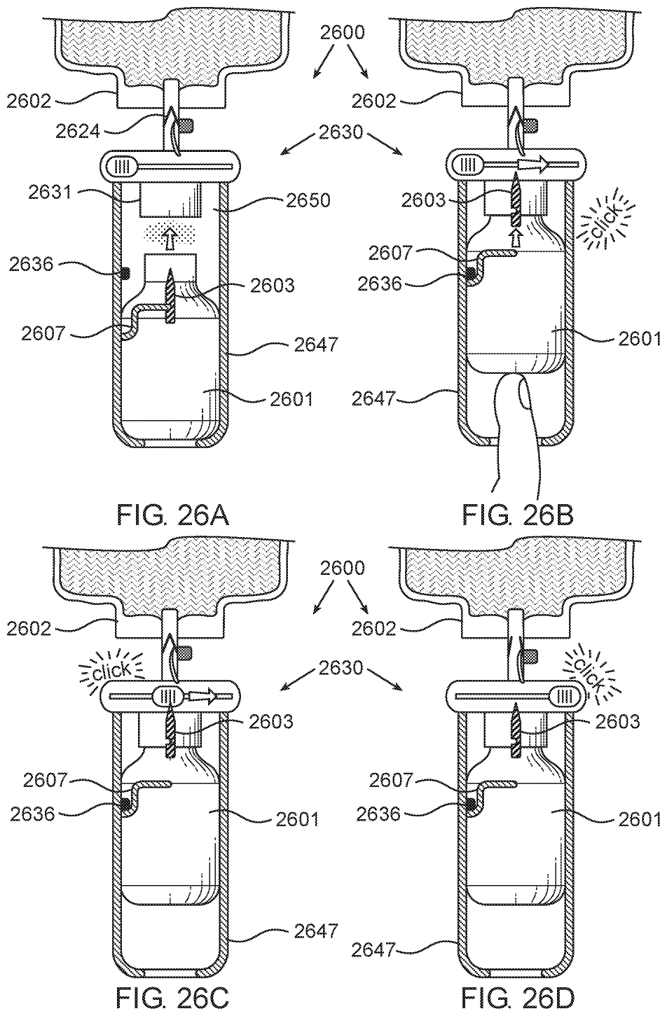

FIGS. 26A-26D illustrate yet another exemplary system 2600 having a decontamination device 2630 which can be coupled with a first vessel, being bottle 2601 and a second vessel, being bag 2602 and can decontaminate the surfaces of one or both the vessels. First vessel 2601 includes piercing member 2603 and a piercing member lock mechanism 2607. Decontamination device 2630 includes downward extending walls/vertical walls 2647 enclosing a lumen 2650 within which vessel 2601 can be placed. Device 2630 further includes a port 2631 for connection to a port of vessel 2601. A piercing member unlock mechanism 2636 extends/protrudes from downward extending walls 2647 into lumen 2650 to allow the unlocking of piercing member 2603. FIG. 26A illustrates vessel 2601 located/disposed within lumen 2650 of the decontamination device 2630. In FIG. 26B, vessel 2601 is connected to port 2631. Piercing member 2603 is unlocked by piercing member unlock mechanism 2636, allowing the piercing member 2603 to pierce through seal/cover of first vessel 2601 and optionally also of device 2630. Wiping member can be actuated by a user to wipe off a surface of vessel 2601 and optionally also of vessel 2602 (FIG. 26C). A fluidic communication between vessels 2601 and 2602 can be established by a user following opening of valve 2624 (FIG. 26D). A “click” sound may be emitted by any of the herein disclosed steps to indicate accomplishment of the step. It is to be noted that steps of allowing an establishment of fluid communication between vessels may performed in a different order or concomitantly with other steps. For example, the step of piercing through a seal or cover may be performed following, before or about the time of wiping off a surface of a vessel. The step of opening a valve may be performed following, before or about the time of wiping off a surface of a vessel and piercing through a seal/cover of a vessel.

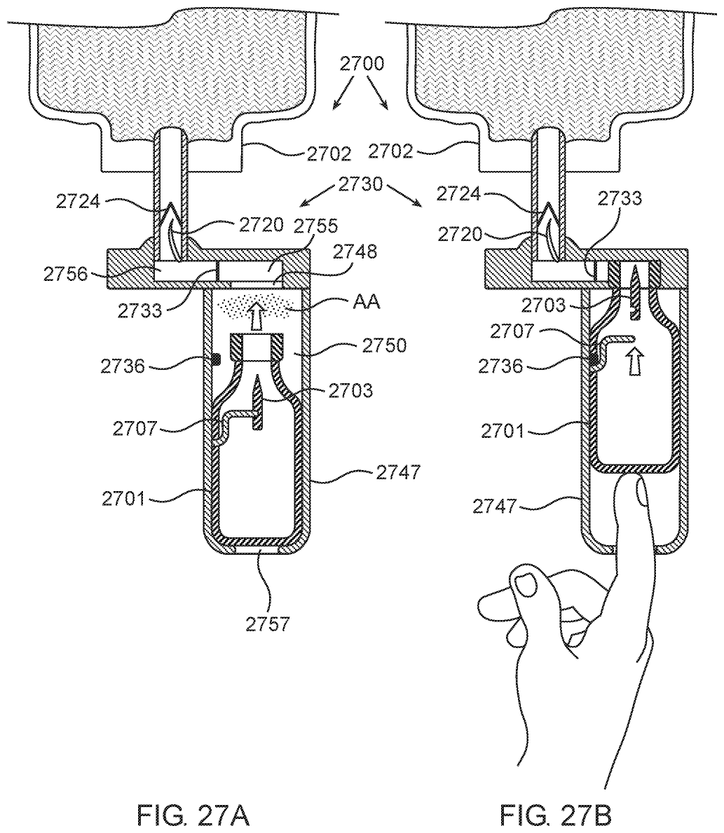

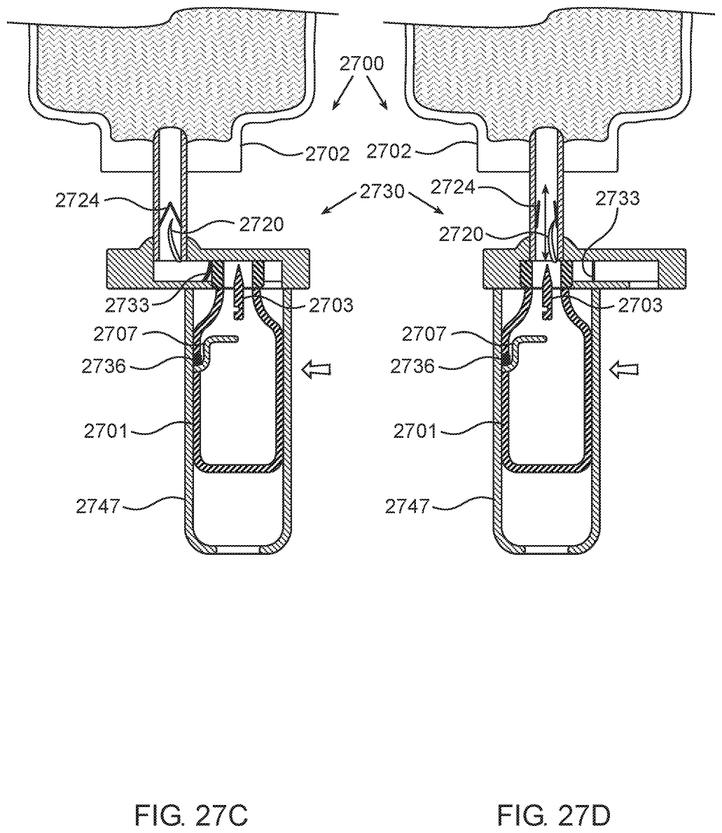

FIGS. 27A-27D illustrate yet another exemplary system 2700 with a decontamination device 2730 having a static wiping member 2733 disposed between a first compartment 2755 and a second compartment 2756 of the device 2730. Vessel 2701 is moveable within the device 2730 between a first position in first compartment 2755 (FIGS. 27B and 27C) and a second position in second compartment 2756 (FIG. 27D). Wiping member 2733 is configured to decontaminate a surface/seal of vessel 2701 upon movement of the vessel 2701 towards second compartment 2756. The system 2700 further includes a valve 2724 disposed within a port of vessel 2702, optionally within/on (not shown) decontamination device 2730. A piercing member 2703 and a lock mechanism thereof 2707 is further incorporated in system 2700. A piercing member unlock mechanism 2736 is provided to unlock lock mechanism 2707. FIG. 27A illustrate vessel 2701 when disposed within a lumen 2750 of device 2730. In FIG. 27B, vessel 2701 is shown connected to device 2730. The connection can be performed by a user which manually pushes the vessel towards device 2730 via aperture 2757 in walls 2747. The piercing member 2703 is unlocked by unlock mechanism 2736 which meets/engages/connects lock mechanism 2707. In FIG. 27C, vessel 2701 moves to second compartment 2756 and undergoes a decontamination by wiping member 2733. In FIG. 27D vessel 2701 is disposed just below or in a longitudinal alignment with vessel 2702 and a fluid communication can be established between the vessels 2701 and 2702. Vessel 2701 is shown entirely disposed inside walls 2747 of the decontamination device 2730 in FIGS. 27C-27D which conceals vessel 2701 from ambient air and prevents escape of any beneficial substance or vapors that may leak from within vessel 2701 during the establishment and/or transfer of the beneficial substance between vessels 2707 and 2702. Vessel 2701 is hermetically sealed within walls 2747 of the decontamination device 2730.

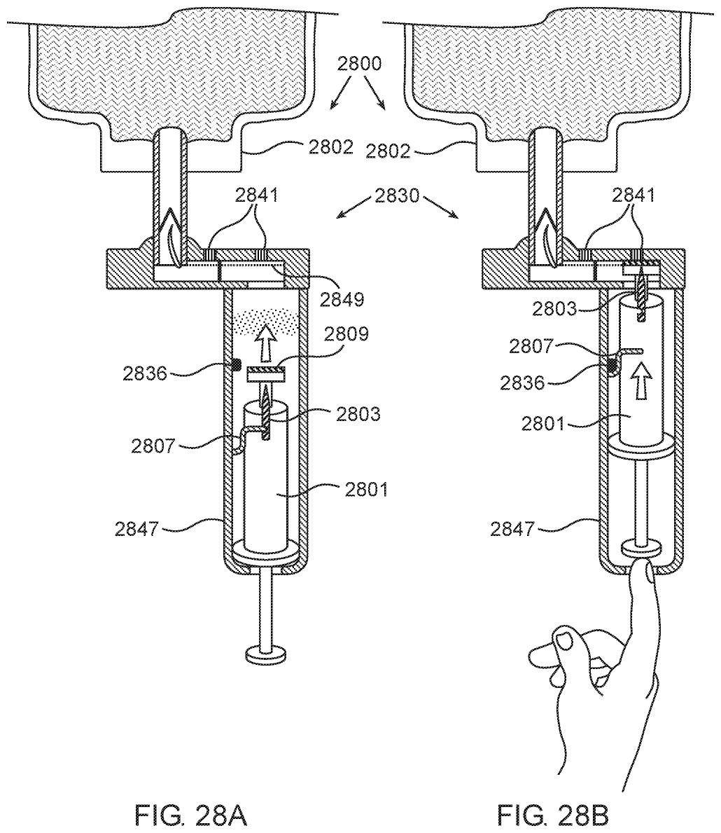

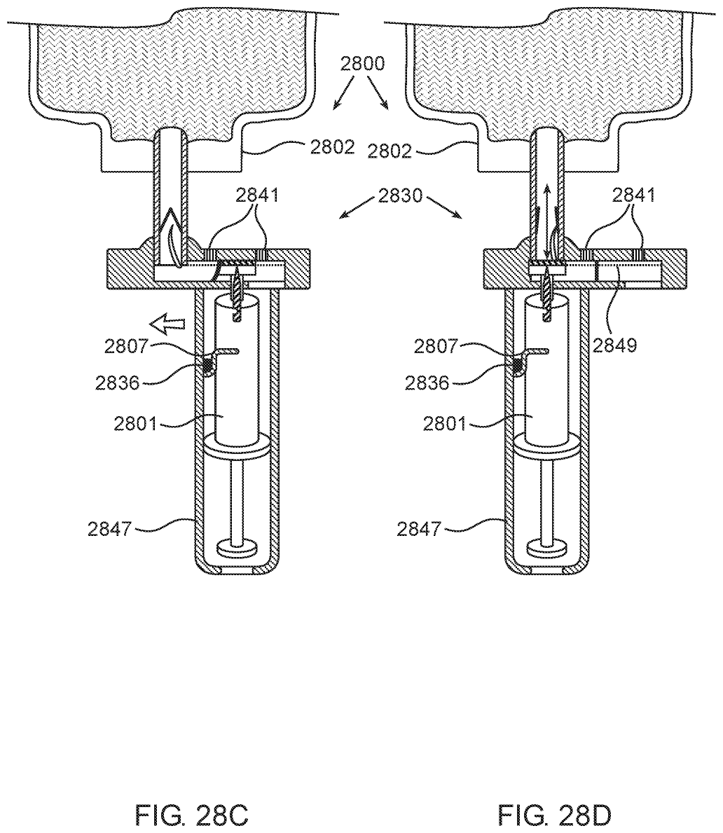

FIGS. 28A-28D illustrate yet another system 2800 wherein first vessel is a syringe 2801 disposed between downward extending walls/vertical walls 2847 and second vessel is a bag 2802. Syringe 2801 has a seal/cover 2809 which can be pierced by piercing member 2803. In system 2800, piercing member lock mechanism 2807 is unlocked by piercing member unlock mechanism 2836 at the time of/following an engagement of syringe 2801 with decontamination device 2830 (FIG. 28B). The movement of vessel 2801 within device 2830 can be mediated via a sliding mechanism, such as rail 2849 disposed on vessel 2801 and/or device 2830. Upon movement of vessel 2801 within device 2830 the seal/cover 2809 can be wiped off by a wiping member disposed inside the device 2830 allowing the decontamination thereof (FIG. 28C). The piercing member 2803 can be actuated to pierce through a seal or cover manually by a user upon pushing plunger towards second vessel 2802 (FIG. 28D). Optionally, or alternately, decontamination device 2830 may have a piercing member (not shown) disposed inside/within/on the decontamination device 2830. An piercing member locking mechanism (not shown) may be disposed within/on the decontamination device 2830 and vessel 2801 may have a piercing member unlock mechanism (not shown) configured to unlock the piercing member (not shown) of the decontamination device 2830 at the time and/or after engagement/coupling of vessel 2801 to vessel 2802.

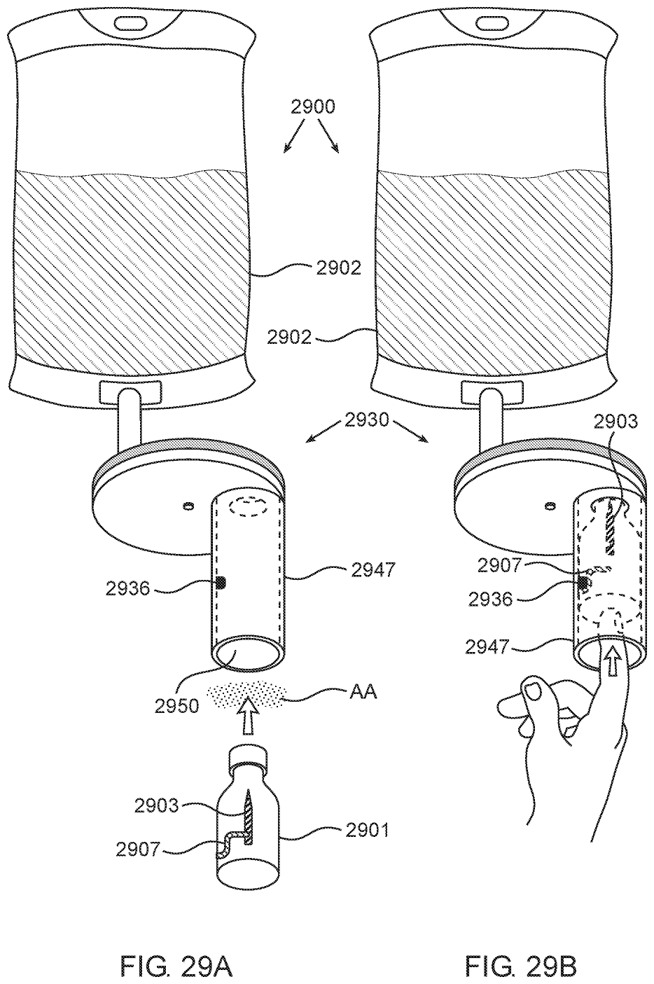

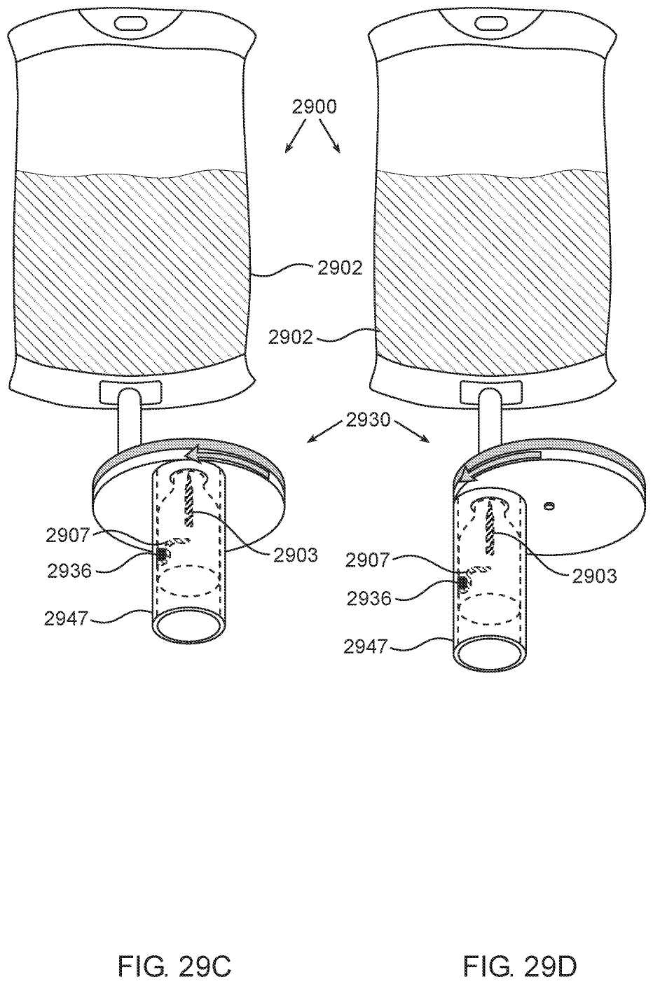

FIGS. 29A-29D illustrate yet another system 2900 which includes a decontamination device in the form of discs 2930. Device 2930 includes downward extending walls 2947 which enclose a lumen 2950 for accommodating therein vessel 2901. The vessel 2901 and device 2930 configured such that vessel 2901 is moveable within the device via rotational movements. A piercing member 2903, and a piercing member lock mechanism 2907 are disposed in/wotjom vessel 2901. When vessel 2901 engages device 2930 via an aperture thereof, the piercing member unlock mechanism 2936 releases piercing member lock mechanism 2907 and thereby piercing member 2903 can be actuated to pierce a seal, optionally manually by pushing vessel 2901 upwards towards second vessel 2902. In one or more embodiments, a wiping member is disposed inside/within and/or between the discs 2930 of the decontamination device. As vessel 2901, optionally a surface of vessel 2901, rotates between various positions within and/or along the discs of the decontamination device 2930 a surface of vessel 2901 is wiped off by the wiping member disposed inside/within/on the decontamination device 2930.

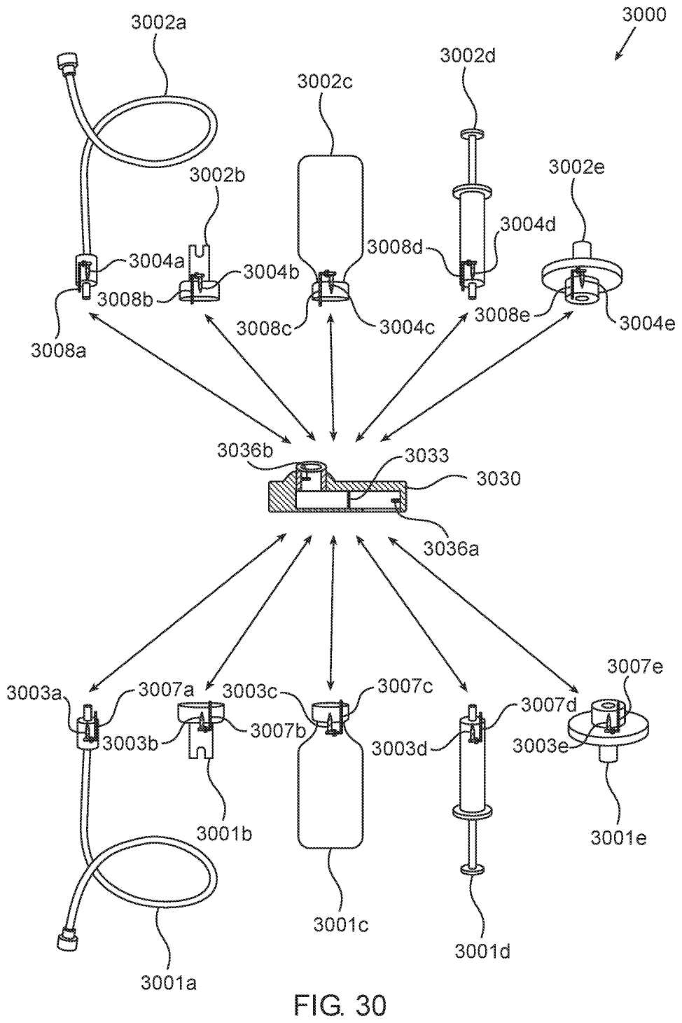

FIG. 30 illustrates system 3000 with a decontamination device, in the form of a connector 3030 that can be engaged with various types of first and second vessels. The device 3030 has a static wiping member 3033 that can decontaminate a surface of the first and/or second vessel upon movement thereof within the device 3030. As illustrated, first vessel may be, for example, a syringe 3001d, a bottle 3001c, a connector 3001b, an infusion line 3001a or a filter 3001e. As illustrated, second vessel may be, for example, a syringe 3002d, a bottle 3002c, a connector 3002b, an infusion line 3002a or a filter 3002e. A portion and/or a surface of the first and/or second vessels are moveable within the connector, thereby effecting decontamination of surfaces of the vessels. A piercing member 3003a-3003e and a piercing member lock mechanism 3007a-3007e is disposed within a port of first vessel, optionally within and/or on the first vessel. A piercing member 3004a-3004e and a piercing member lock mechanism 3008a-3008e is disposed within a port of second vessel. A piercing member unlock mechanism 3036a and 3036b may be disposed in decontamination device 3030 to unlock piercing member and/or piercing member lock mechanism of the vessels.

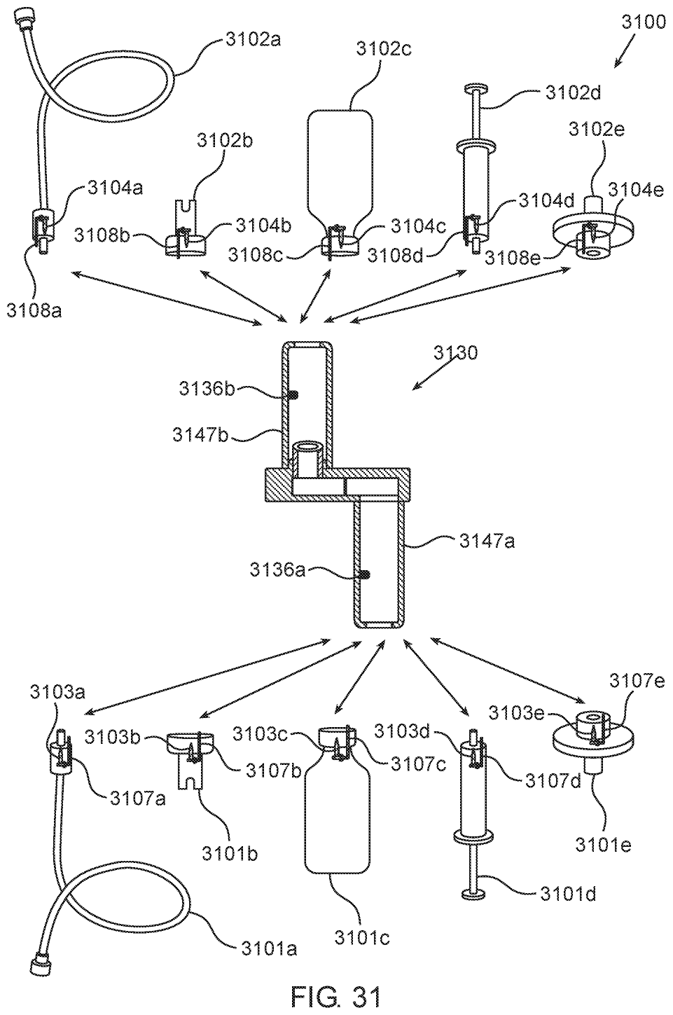

FIG. 31 illustrates yet a similar system 3100 which similarly includes optional first vessel, i.e., vessels 3101a-3101e and second vessels 3102a-3102e which can be coupled to connector 3130. The device 3130 include vertical walls 3147a and 3147b for accommodating therein a first vessel and a second vessel. respectively. A piercing member 3103a-3103e and a piercing member lock mechanism 3107a-3107e is disposed within a port of first vessel. A piercing member 3104a-3104e and a piercing member lock mechanism 3108a-3108e is disposed within a port of second vessel. A piercing member unlock mechanism 3136a and 3136b may be disposed in decontamination device 3130 to unlock piercing member lock and lock mechanism of the vessels.

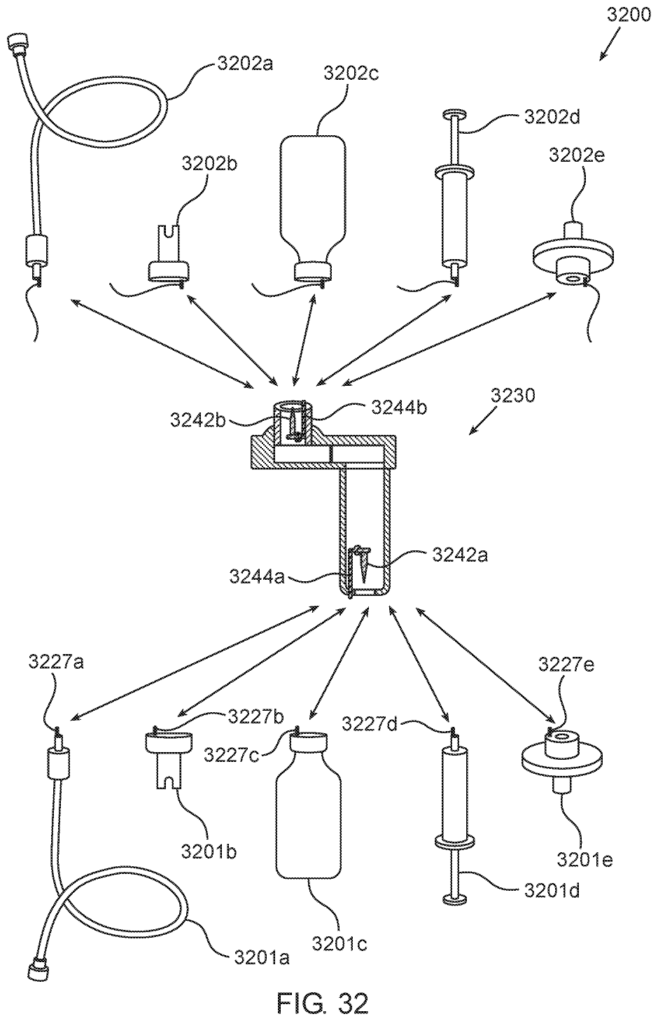

FIG. 32 illustrates yet a similar system 3200 which similarly includes optional first vessel, i.e., vessels 3201a-3201e and second vessels 3202a-3202e which can be coupled to connector 3230. The device 3230 includes piercing member 3242a, 3242b and a piercing member lock mechanism 3244a, 3244b disposed within decontamination device 3230.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for the engagement and establishment of fluid passageway between vessels, the system comprises:
a first vessel; and
a second vessel configured to be coupled to the first vessel;
wherein at least one of the first and second vessels comprises a piercing member, the piercing member disposed within the vessel in a retracted position and configured to pierce through a cover/seal of the vessel at about the time of or following the engagement between the first and second vessels, thereby establishing a fluid transfer between the vessels.

2. The system of claim 1, wherein the piercing member is configured to pierce through a cover/seal of both the first and second vessels at about the time of or following the engagement between the first and second vessels.

3. The system of claim 1, further comprising a piercing member lock mechanism connected to the piercing member and configured to maintain the piercing member in the retracted position prior to an engagement between the vessels, thereby preventing accidental fluid spillage from the vessel.

4. The system of claim 3, wherein the piercing member lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

5. The system of claim 1, further comprising a piercing member unlock mechanism configured to unlock/allow movement of the piercing member, and wherein the unlock mechanism unlocks the piercing member at about the time of or following the engagement between the first and second vessels.

6. The system of claim 3, further comprising a piercing member unlock mechanism configured to engage/connect the lock mechanism and unlock thereof, thereby allow the movement of the piercing member, and wherein the unlock mechanism unlocks the piercing member lock mechanism at about the time of or following the engagement between the first and second vessels.

7. The system of claim 5, wherein the piercing member unlock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

8. The system of claim 1, wherein the piercing member pierces through a seal/cover of a vessel via a push motion, a twist motion, a turn motion or a combination thereof between the first and second vessels.

9. The system of claim 1, further comprising an engagement mechanism to connect the first vessel to the second vessel.

10. The system of claim 9, wherein the engagement mechanism is selected from a thread, a luer, a ratchet teeth mechanism, a retention member, an adhesive mechanism, a clamping member and combinations thereof.

11. The system of claim 1, further comprising a valve within one or both vessels to monitor the fluid transfer between the first and/or second vessel(s).

12. The system of claim 11, further comprising a valve lock mechanism and optionally a valve unlock mechanism to prevent accidental fluid spillage from a vessel.

13. The system of claim 1, further comprising a plunger disposed on the first and/or second vessel(s), the plunger configured to allow for the fluid transfer between the first vessel and the second vessel.

14. The system of claim 13, further comprising a plunger lock mechanism configured to lock a plunger and prevent fluid transfer between the first vessel and second vessels prior to an engagement between the vessels.

15. The system of claim 1, wherein the vessel is selected from the group consisting of a bottle, a syringe, an infusion line, a connector, a filter, a manifold, a bag port, a bottle port, a vial port, a cartridge and a combination thereof.

16. The system of claim 1, further comprising a decontamination device configured to be coupled to the first vessel and second vessel and allow the fluid passageway therebetween, the decontamination device configured to decontaminate a surface of at least one of the first and second vessels.

17. The system of claim 16, further comprising an engagement mechanism to connect the decontamination device to the first and/or second vessel.

18. The system of claim 16, wherein the vessel and/or decontamination device includes a port having peripheral vertical walls enclosing a lumen, the lumen configured to receive or engage with a vessel and/or decontamination device, or a portion thereof.

19. The system of claim 16, wherein the decontamination device is pre-attached to the first or second vessels.

20. The system of claim 16, wherein the decontamination device is integrally attached to the first or second vessel.

21. The system of claim 16, wherein the decontamination device includes a wiping member configured to wipe off a surface of a vessel.

22. The system of claim 21, wherein the first and/or second vessel is configured to move within the device and wherein the wiping member configured to remove contaminants from a surface of said first and/or second vessels at about the time of said vessel's movement.

23. The system of claim 21, wherein the wiping member is configured to move within the device such to allow decontamination of a surface of at least one of said first and second vessels at about the time of said wiping member's movement.

24. The system of claim 21, wherein the decontamination device includes a sliding mechanism located on and/or attached to said wiping member and/or said vessel and configured to allow for the movement of said wiping member and/or said vessel within said device.

25. The system of claim 16, further comprising a piercing member disposed within the decontamination device and configured to pierce through a seal or cover of a vessel and/or port of the decontamination device and prevent the premature fluid transfer between the first vessel and the second vessel and/or fluid spillage, prior to an engagement between the first and/or second vessels and the decontamination device.
